(19) United States
(12) Patent Application Publication
(10) Pub. No.: US 2026/0016488 A1
(43) Pub. Date: Jan. 15, 2026

(54) CIRCULATING PROTEIN BIOMARKERS OF NECROTIZING ENTERROCOLITIS

(71) Applicant: The Regents of the University of Colorado, A Body Corporate, Denver, CO (US)

(72) Inventor: Jesse Davidson, Denver, CO (US)

(21) Appl. No.: 19/264,069

(22) Filed: Jul. 9, 2025

Related U.S. Application Data
(60) Provisional application No. 63/668,982, filed on Jul. 9, 2024.

(51) Int. Cl. G01N 33/68

(52) U.S. Cl. CPC G01N 33/6893; G01N 2800/38

(57) ABSTRACT

Disclosed herein are methods, devices, and systems for accurately detecting and/or diagnosing a child having or at risk of developing necrotizing enterocolitis (NEC). In most embodiments, the child is currently suffering from congenital heart disease (CHD). The disclosed methods, devices, and systems may be useful in monitoring the administration and/or response to therapy for CHD and/or NEC. The disclosed methods, devices, and systems may involve detecting one or more biomarkers in a sample from the child, for example a plasma sample, wherein the sample is less than or equal to about 20 microliters.

### CROSS-REFERENCE TO RELATED APPLICATIONS

This application claims benefit of priority pursuant to 35 U.S.C. § 119 (c) of U.S. provisional patent application No. 63/668,982 entitled “CIRCULATING PROTEIN BIOMARKERS OF NECROTIZING ENTERROCOLITIS,” filed on 9 Jul. 2024, which is hereby incorporated by reference in its entirety.

### BACKGROUND

Congenital heart disease (CHD) is a risk factor for the development of necrotizing enterocolitis (NEC). NEC has an incidence of 3-5% in the CHD population and is associated with a 2-fold increase in mortality (24.4% vs 11.8% in CHD neonates without NEC), and a 3-fold increase in length of stay (54 vs 18 days). Although poorly understood, cardiac NEC appears to be a distinct entity from classic NEC of the premature infant. Biomarkers capable of predicting NEC in CHD patients are lacking. Specifically, there are no clinically available biomarkers to 1) diagnose NEC before clinical symptoms occur, 2) provide prognostic information to clinicians and families, 3) determine effectiveness of treatment/response to therapy, or 4) identify different etiologies (e.g., ischemic vs allergic) that require personalized treatments.

### SUMMARY

Disclosed herein are methods, devices, and systems useful for diagnosing or predicting NEC comprising: two or more molecules recognizing a biomarker, wherein the biomarker is one or more of LRRN1, VASHI, NELL2, DNAJB8, IGFBP1, PTPRN2, AFP, CD74, BST2, CD99, KIR3DL1, RBP2, CES2, NCS1, C4BPB, RAB6A, CCL4, FABP2, FUCA1, REG3A, GGA1, ATXN10, MUC13, ODAM, ARID4B, CCL2, CLEC11A, ITGAM, INPP1, ICOSLG, LRP1, MMP12, GBP2, GZMB, EPO, CXCL9, LGALS4, VIM, GZMH, MFAP5, CD46, SSB, CTSS, JAM2, KIR2DL3, BGN, PDGFC, RAD23B, FAM3B, GALNT7, VATI, HBEGF, MIA, TPT1, ILIR1, CD83, MLN, CD200, CLEC4D, CLEC4C, CXLC10, BTN2A1, TFF2, CTRC, EPHX2, CHEK2, MFAP3, DCN, LGALS1, THBD, CDH1, FCN2, IL6R, ILKAP, FOSB, MASP1, DRAXIN, ABHD14B, CD274, ADAM8, SCARF 2, RGMA, DSC2, AKT3, NTF4, TBLX1, LYPD8, LYAR, ITGB1BP1, GH2, EPHA2, LRRC25, ALPP, LAG3, KLK4, CD300LF, HBQ1, FURIN, FLT4, FGFR2, NECTIN4, P4HB, ACTN4, WAS, HLA-E, PARP1, NT5C3A, CASP2, OSM, HSD11B1, SLAMF7, IL32, CCL3, TRIM21, TRAF2, ALDH3A1, EDAR, KLRB1, DFFA, TGFB1, REG4, CXCL1, CEP43, IRAG2, TP53INP1, GLO1, GRK5, PDGFRA, SDC4, PRKAR1A, FADD, GDF2, FAM3C, CASP3, MB, PAM, SERPINE1, TGFBI, ITGB1, CD93, ATP6VIF, BAX, NDRG1, SIRT5, MDGA1, STAMBP, EREG, IMPA1, CRADD, TNR, NPM1, PRDX1, PSME1, LAYN, CDH15, VTA1, NSFL1C, MSR1, TFF1, CLEC14A, bin2, PARK7, CCT5, SFTPA2, CRACR2A, SUGT1, CHAC2, GNE, FMR1, CDC27, CEP20, TBC1D23, UBAC1, OMG, VPS37A, TMPRSS15, KIFBP, CD207, CNPY4, CALCOCO1, CPE, METAP2, PPY, CDC37, FGFBP1, FGF5, SMPDL3A, NPPB, AMY2B, GUSB, ICAM2, CELA3A, PROC, PAEP, and ASAH2. In many embodiments, the biomarker is one or more of LRRN1, VASH1, NELL2, DNAJB8, IGFBP1, PTPRN2, AFP, CD74, BST2, CD99, KIR3DL1, RBP2, CES2, NCS1, C4BPB, RAB6A, CCL4, FABP2, FUCA1, REG3A, GGA1, ATXN10, MUC13, ODAM, ARID4B, CCL2, CLEC11A, ITGAM, INPP1, ICOSLG, LRP1, and MMP12, for example iFAB/FABP2, and/or the molecule may be selected from a nucleic acid, a peptide, or a combination thereof. Also disclosed are devices useful for diagnosing or predicting NEC comprising, wherein the molecule may be in solution or covalently attached to a surface of the device.

Further, methods for detecting one or more biomarkers are disclosed, comprising: contacting a sample with at least one molecule having affinity for the the biomarker, the biomarker selected from one or more of LRRN1, VASH1, NELL2, DNAJB8, IGFBP1, PTPRN2, AFP, CD74, BST2, CD99, KIR3DL1, RBP2, CES2, NCS1, C4BPB, RAB6A, CCL4, FABP2, FUCA1, REG3A, GGA1, ATXN10, MUC13, ODAM, ARID4B, CCL2, CLEC11A, ITGAM, INPP1, ICOSLG, LRP1, MMP12, GBP2, GZMB, EPO, CXCL9, LGALS4, VIM, GZMH, MFAP5, CD46, SSB, CTSS, JAM2, KIR2DL3, BGN, PDGFC, RAD23B, FAM3B, GALNT7, VAT1, HBEGF, MIA, TPT1, IL1R1, CD83, MLN, CD200, CLEC4D, CLEC4C, CXLC10, BTN2A1, TFF2, CTRC, EPHX2, CHEK2, MFAP3, DCN, LGALS1, THBD, CDH1, FCN2, IL6R, ILKAP, FOSB, MASP1, DRAXIN, ABHD14B, CD274, ADAM8, SCARF 2, RGMA, DSC2, AKT3, NTF4, TBLX1, LYPD8, LYAR, ITGB1BP1, GH2, EPHA2, LRRC25, ALPP, LAG3, KLK4, CD300LF, HBQ1, FURIN, FLT4, FGFR2, NECTIN4, P4HB, ACTN4, WAS, HLA-E, PARP1, NT5C3A, CASP2, OSM, HSD11B1, SLAMF7, IL32, CCL3, TRIM21,TRAF2, ALDH3A1, EDAR, KLRB1, DFFA, TGFB1, REG4, CXCL1, CEP43, IRAG2, TP53INP1, GLO1, GRK5, PDGFRA, SDC4, PRKAR1A, FADD, GDF2, FAM3C, CASP3, MB, PAM, SERPINE1, TGFB1, ITGB1, CD93, ATP6V1F, BAX, NDRG1, SIRT5, MDGA1, STAMBP, EREG, IMPA1, CRADD, TNR, NPM1, PRDX1, PSME1, LAYN, CDH15, VTA1, NSFL1C, MSR1, TFF1, CLEC14A, bin2, PARK7, CCT5, SFTPA2, CRACR2A, SUGT1, CHAC2, GNE, FMR1, CDC27, CEP20, TBC1D23, UBAC1, OMG, VPS37A, TMPRSS15, KIFBP, CD207, CNPY4, CALCOCO1, CPE, METAP2, PPY, CDC37, FGFBP1, FGF5, SMPDL3A, NPPB, AMY2B, GUSB, ICAM2, CELA3A, PROC, PAEP, and ASAH2. In some embodiments, the biomarker is one or more of wherein the biomarker is selected from LRRN1, VASHI, NELL2, DNAJB8, IGFBP1, PTPRN2, AFP, CD74, BST2, CD99, KIR3DL1, RBP2, CES2, NCS1, C4BPB, RAB6A, CCL4, FABP2, FUCA1, REG3A, GGA1, ATXN10, MUC13, ODAM, ARID4B, CCL2, CLEC11A, ITGAM, INPP1, ICOSLG, LRP1, and MMP12, in many cases, the biomarker is iFAB and/or the amount of biomarker or molecule correlates to a signal. In many embodiments, the signal of the molecule/biomolecule in the sample may be higher or may be lower than a signal in a control sample, for one example a sample collected earlier. In many embodiments, the earlier collected sample is collected before and/or at or near the time of surgical treatment for CHD.

Also disclosed are methods of for diagnosing or predicting NEC in a patient at risk thereof, comprising: obtaining a sample of circulating proteins from the patient; identifying the presence of one or more biomarkers in the sample to determine a signature; and comparing the patient biomarker signature to a diagnostic and/or predictive signature to determine the presence of NEC and/or risk of developing NEC, wherein the biomarker is one or more of LRRN1, VASH1, NELL2, DNAJB8, IGFBP1, PTPRN2, AFP, CD74, BST2, CD99, KIR3DL1, RBP2, CES2, NCS1, C4BPB, RAB6A, CCL4, FABP2, FUCA1, REG3A, GGA1, ATXN10, MUC13, ODAM, ARID4B, CCL2, CLEC11A, ITGAM, INPP1, ICOSLG, LRP1, MMP12, GBP2, GZMB, EPO, CXCL9, LGALS4, VIM, GZMH, MFAP5, CD46, SSB, CTSS, JAM2, KIR2DL3, BGN, PDGFC, RAD23B, FAM3B, GALNT7, VAT1, HBEGF, MIA, TPT1, IL1R1, CD83, MLN, CD200, CLEC4D, CLEC4C, CXLC10, BTN2A1, TFF2, CTRC, EPHX2, CHEK2, MFAP3, DCN, LGALS1, THBD, CDH1, FCN2, IL6R, ILKAP, FOSB, MASP1, DRAXIN, ABHD14B, CD274, ADAM8, SCARF 2, RGMA, DSC2, AKT3, NTF4, TBLX1, LYPD8, LYAR, ITGB1BP1, GH2, EPHA2, LRRC25, ALPP, LAG3, KLK4, CD300LF, HBQ1, FURIN, FLT4, FGFR2, NECTIN4, P4HB, ACTN4, WAS, HLA-E, PARP1, NT5C3A, CASP2, OSM, HSD11B1, SLAMF7, IL32, CCL3, TRIM21, TRAF2, ALDH3A1, EDAR, KLRB1, DFFA, TGFB1, REG4, CXCL1, CEP43, IRAG2, TP53INP1, GLO1, GRK5, PDGFRA, SDC4, PRKAR1A, FADD, GDF2, FAM3C, CASP3, MB, PAM, SERPINE1, TGFBI, ITGB1, CD93, ATP6V1F, BAX, NDRG1, SIRT5, MDGA1, STAMBP, EREG, IMPA1, CRADD, TNR, NPM1, PRDX1, PSME1, LAYN, CDH15, VTA1, NSFL1C, MSR1, TFF1, CLEC14A, bin2, PARK7, CCT5, SFTPA2, CRACR2A, SUGT1, CHAC2, GNE, FMR1, CDC27, CEP20, TBC1D23, UBAC1, OMG, VPS37A, TMPRSS15, KIFBP, CD207, CNPY4, CALCOCO1, CPE, METAP2, PPY, CDC37, FGFBP1, FGF5, SMPDL3A, NPPB, AMY2B, GUSB, ICAM2, CELA3A, PROC, PAEP, and ASAH2. In many embodiments, the biomarker is one or more of wherein the biomarker is selected from LRRN1, VASH1, NELL2, DNAJB8, IGFBP1, PTPRN2, AFP, CD74, BST2, CD99, KIR3DL1, RBP2, CES2, NCS1, C4BPB, RAB6A, CCL4, FABP2, FUCA1, REG3A, GGA1, ATXN10, MUC13, ODAM, ARID4B, CCL2, CLEC11A, ITGAM, INPP1, ICOSLG, LRP1, and MMP12, for one example iFAB. In many embodiments, the patient may have been treated for CHD previous to having obtained the sample.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIG. 1A is a table listing subject characteristics.

FIG. 1B is a comparison of iFABP levels on NEC day 1 (green) vs controls (red). Comparison of iFABP levels on NEC day 1 (green) vs day 5 (blue). Red arrow=trajectory of iFABP in the single nonsurvivor.

FIG. 1C is a volcano plot analysis of differentially expressed proteins between NEC patients (NEC day 1) and controls; red: ≥2-fold increase (P<0.1) in NEC cases vs controls, blue: ≥50% decrease (P<0.1) in NEC cases compared with controls. Significant proteins are annotated with their HUGO Gene Nomenclature.

FIG. 1D is a diagram of protein clusters showing extensive immune system upregulation (blue), angiogenesis and endothelial migration (green), and disruption in proteins involved in maintaining the gastrointestinal epithelium (red). AVSD indicates atrioventricular septal defect; DORV, double outlet right ventricle; HLHS, hypoplastic left heart syndrome; iFABP, intestinal fatty acid binding protein; NPX, normalized protein expression (log 2 scale); TAPVR, total anomalous pulmonary venous return; TGA, transposition of the great arteries; and VSD, ventricular septal defect.

FIG. 2 is a scores plot showing PLS-DA demonstrates a substantial shift in the global proteome at 2 hrs postoperatively with incomplete recovery by 48 hrs.

FIG. 3 is a scores plot showing that PLS-DA demonstrates the ability to differentiate among tiers of vasoactive inotropic support at 48 hrs postoperatively based on the global 2 hr circulating proteome.

FIG. 4 is a bar graph showing IFABP levels (ng/ml) at 6 and 24 hrs postoperatively in infants who subsequently developed NEC compared to those who did not. Suspected NEC=Bell Stage 1, Definite NEC=Bell Stage 2 or 3.

FIG. 5 is a scores plot showing partial least squares discriminant analysis demonstrating distinct proteomic profiles of interstage infants with single ventricle heart disease and subsequent upper quartile (red) vs. lower quartile (green) BSID-4 comprehensive motor scores at lyr of age.

### DETAILED DESCRIPTION

Disclosed herein are compositions, devices, methods, and systems for diagnosing, prognosing, and/or monitoring neonates for development of necrotizing enterocolitis (NEC). Disclosed herein are biomarkers that may be useful in same.

Applicants performed proteomic analysis of high-risk children with congenital heart disease who underwent neonatal surgical repair. In this cohort, the circulating proteome of neonates who developed NEC was evaluated and compared it to those who did not. Applicants also then evaluated the daily changes in the circulating proteome of neonates with NEC to determine the changes in the proteome that occurred over the first 5 days of therapy. 195 proteins (listed below) were identified that distinguished between neonates with and without NEC with an area under the curve (AUC)>0.85. Of these, 32 demonstrated excellent discrimination with an AUC>0.95. The proteins identified belong to 3 primary biologic systems: maintenance of gastrointestinal epithelium, inflammation, and angiogenesis/endothelial migration. Furthermore, the majority of these biomarkers demonstrated a response to therapy and accurately identified the child who did not respond to therapy and ultimately died 10 days after their NEC diagnosis. In this invention, Applicants disclose that these 195 biomarkers (alone and/or in a combined panel) can help accurately diagnose NEC in children with CHD, assist clinicians with prognostic assessment, and be used as a tool to monitor the response to therapy. Protein biomarkers disclosed are selected from (HUGO nomenclature, with bolding used to identify downregulated biomarkers):
AUC 1.0: LRRN1, VASH1, NELL2, DNAJB8, IGFBP1, PTPRN2, AFP, CD74, BST2, CD99, KIR3DL1, RBP2, CES2, NCS1, C4BPB;
AUC 0.95-1.0: RAB6A, CCL4, FABP2, FUCA1, REG3A, GGA1, ATXN10, MUC13, ODAM, ARID4B, CCL2, CLEC11A, ITGAM, INPP1, ICOSLG, LRP1, MMP12;
AUC 0.9-0.95: GBP2, GZMB, EPO, CXCL9, LGALS4, VIM, GZMH, MFAP5, CD46, SSB, CTSS, JAM2, KIR2DL3, BGN, PDGFC, RAD23B, FAM3B, GALNT7, VAT1, HBEGF, MIA;
and
AUC 0.85-0.9: TPT1, IL1R1, CD83, MLN, CD200, CLEC4D, CLEC4C, CXLC10, BTN2A1, TFF2, CTRC, EPHX2, CHEK2, MFAP3, DCN, LGALS1, THBD, CDH1, FCN2, IL6R, ILKAP, FOSB, MASP1, DRAXIN, ABHD14B, CD274, ADAM8, SCARF 2, RGMA, DSC2, AKT3, NTF4, TBLX1, LYPD8, LYAR, ITGB1BP1, GH2, EPHA2, LRRC25, ALPP, LAG3, KLK4, CD300LF, HBQ1, FURIN, FLT4, FGFR2, NECTIN4, P4HB, ACTN4, WAS, HLA-E, PARP1, NT5C3A, CASP2, OSM, HSD11B1, SLAMF7, IL32, CCL3, TRIM21, TRAF2, ALDH3A1, EDAR, KLRB1, DFFA, TGFB1, REG4, CXCL1, CEP43, IRAG2, TP53INP1, GLO1, GRK5, PDGFRA, SDC4, PRKAR1A, FADD, GDF2, FAM3C, CASP3, MB, PAM, SERPINE1, TGFB1, ITGB1, CD93, ATP6V1F, BAX, NDRG1, SIRT5, MDGA1, STAMBP, EREG, IMPA1, CRADD, TNR, NPM1, PRDX1, PSME1, LAYN, CDH15, VTA1, NSFL1C, MSR1, TFF1, CLEC14A, bin2, PARK7, CCT5, SFTPA2, CRACR2A, SUGT1, CHAC2, GNE, FMR1, CDC27, CEP20, TBC1D23, UBAC1, OMG, VPS37A, TMPRSS15, KIFBP, CD207, CNPY4, CALCOCO1, CPE, METAP2, PPY, CDC37, FGFBP1, FGF5, SMPDL3A, NPPB, AMY2B, GUSB, ICAM2, CELA3A, PROC, PAEP, ASAH2. Protein biomarkers and molecules recognizing same, as disclosed herein, may be full-length or fragments thereof.

Currently there are no diagnostic, prognostic, or therapeutic monitoring biomarkers available to assist clinicians with the care of neonates who develop NEC. Therefore, most neonates and infants who develop symptoms are treated with the same basic therapies including cessation of enteral nutrition and initiation of broad-spectrum intravenous antibiotics. Clinical biomarkers would allow: i. Early diagnosis: allowing initiation of therapy prior to clinical deterioration, including slower introduction of enteral nutrition in cases with early low-level biomarker changes without clinical symptoms. ii. Accurate diagnosis: limiting enteral nutrition interruptions and unnecessary antibiotic exposure in cases with equivocal symptoms and negative biomarker screening. iii. Accurate therapy: identifying the distinct molecular phenotypes of NEC versus allergic colitis allowing early transition to hypoallergenic formula when indicated. iv. Evaluation of response to therapy: allowing early determination of effectiveness of therapy leading to the ability to change or augment treatment (e.g., addition of inotropic support) in cases where the biomarker profile is not improving. Furthermore, a panel including mixed markers of gastrointestinal epithelial repair, inflammation, and angiogenesis targets all aspects of this pathophysiology in a superior fashion to single biomarkers alone.

Congenital heart disease (CHD) is a risk factor for the development of necrotizing enterocolitis (NEC).1 NEC has an incidence of 3-5% in the CHD population and is associated with a 2-fold increase in mortality (24.4% vs 11.8% in CHD neonates without NEC), and a 3-fold increase in length of stay (54 vs 18 days). Although poorly understood, cardiac NEC appears to be a distinct entity from classic NEC of the premature infant. Biomarkers capable of predicting NEC in CHD patients are lacking. One candidate is intestinal fatty acid binding protein (IFABP/FABP2). IFABP is highly expressed in the intestinal epithelium and is involved in lipid metabolism, control of inflammation, cell proliferation, and barrier function. Circulating IFABP increases immediately after infant cardiac surgery and higher immediate postoperative levels are associated with increased risk of subsequent NEC. IFABP's utility as a biomarker for the detection of acute NEC or the response to treatment has not been established. Furthermore, the systemic response to NEC is poorly defined. This pilot study sought to evaluate changes in IFABP levels and the circulating proteome associated with the onset of NEC and the natural history of IFABP levels during treatment.

This pilot study is part of a larger parent study on the metabolic response to CHD surgery (R01HL156936). Neonates undergoing cardiothoracic surgery with cardiopulmonary bypass were prospectively enrolled. NEC was defined as Bell's staging ≥1A/B. Plasma samples (10-40 μL) were collected from routine clinical chemistries on the first day of acute NEC symptoms and on day 5 of treatment (NEC days 1 and 5 respectively). Treatment was based on local NEC clinical protocols. For patients without a NEC diagnosis, remnant plasma samples from postoperative day (POD) 7 served as controls. Samples were stored at −80° C. for batch analysis. Proteomic analysis of 1,536 proteins (including IFABP) was performed using Olink Explore (Olink, Uppsala, Sweden). Proteins were log 2 transformed for analysis. The primary outcome was the difference in IFABP levels between NEC patients on NEC day 1 and controls on POD 7 (t-test; significance p<0.05). Secondary outcomes included change in IFABP from NEC day 1-5 and exploratory proteomic analysis to identify proteins demonstrating at least a 2-fold difference between NEC/no NEC patients at a p-value<0.1. Proteins meeting the outlined statistical significance were then expanded using GenePlexus, a network-based protein discovery tool. The resulting proteins were entered into Metascape for exploration by tissue of origin, and STRING for protein-protein network analysis.

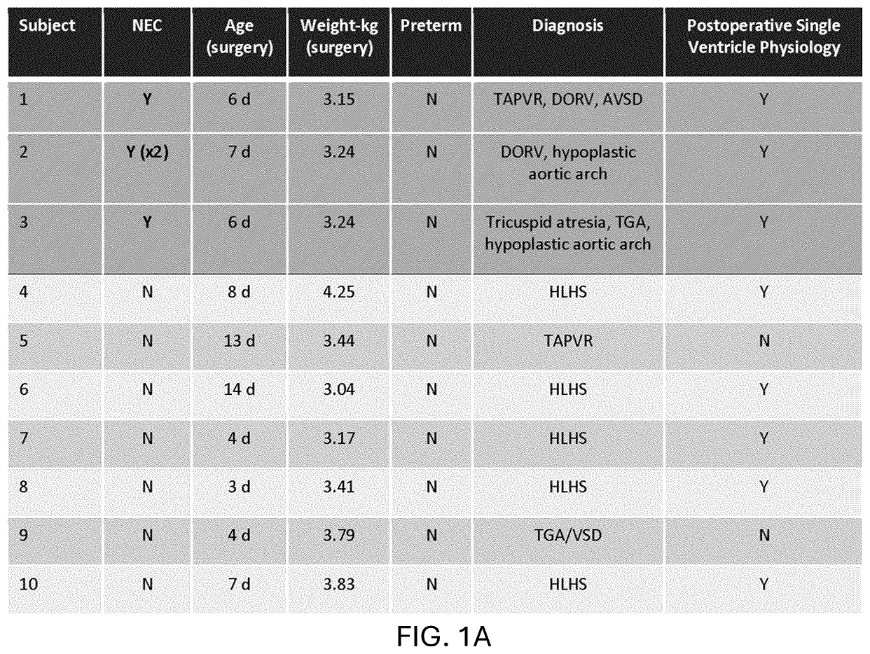

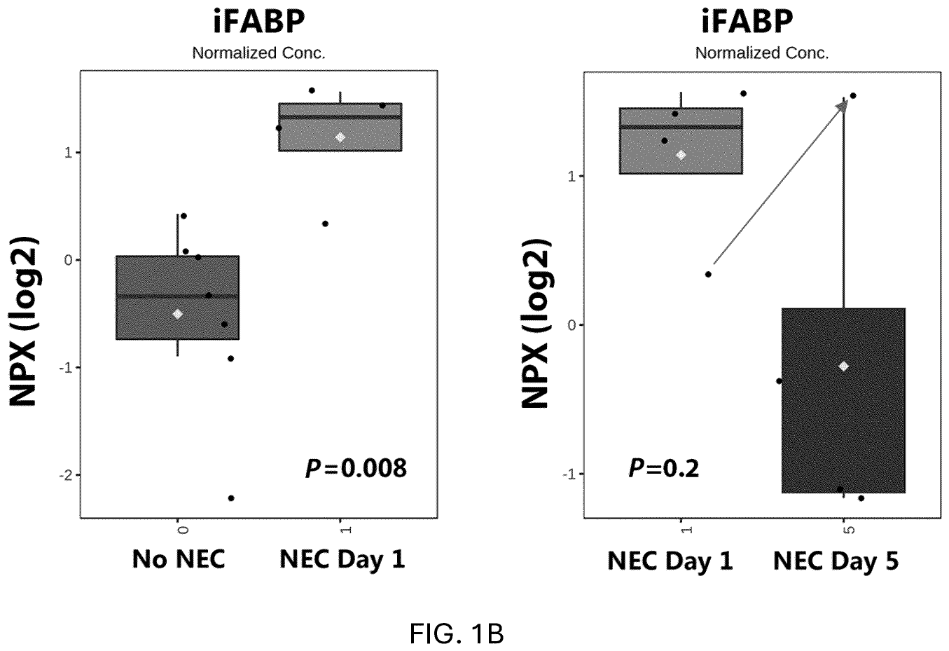

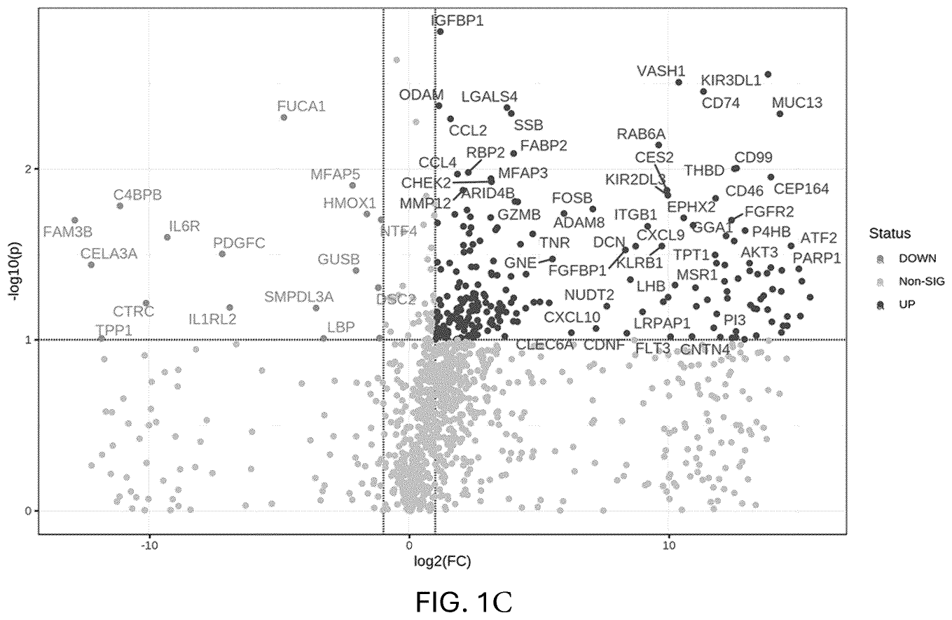

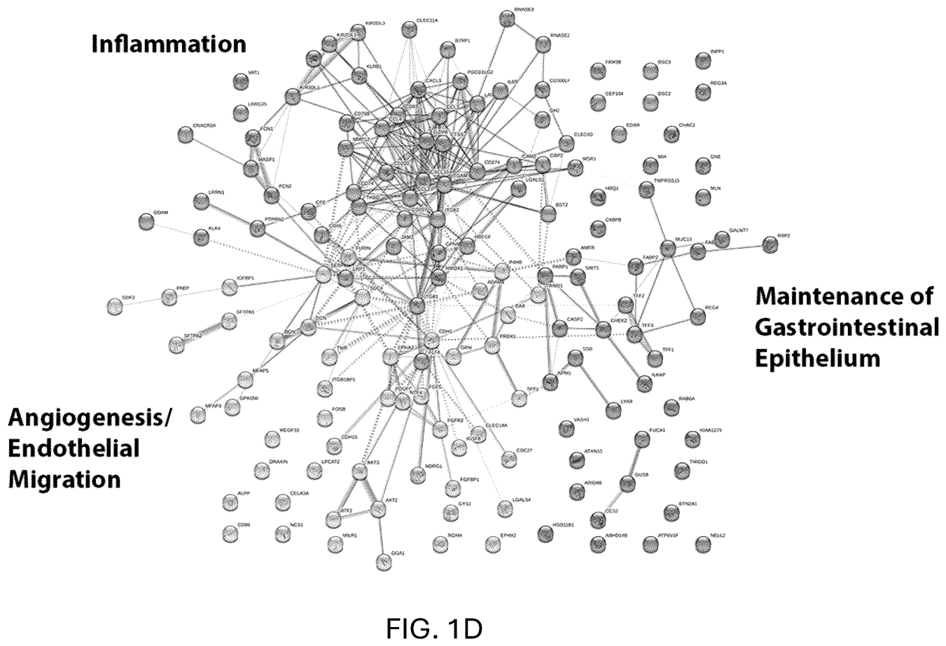

Ten neonates with comparable high-risk cardiac surgeries were included (subject characteristics shown in FIG. 1A). Three subjects developed NEC (PODs 20, 33, and 34) with one subject experiencing a second episode of NEC (POD 50). Seven subjects did not develop NEC. IFABP levels at the onset of NEC were significantly higher compared to the non-NEC group. IFABP levels then trended down between NEC day 1 and day 5, except for one subject who maintained high IFABP levels at day 5 and subsequently died ten days later (FIG. 1B). Proteomic analysis identified 246 proteins that were significantly dysregulated (FIG. 1C). Of these proteins, 229 were upregulated in NEC patients while 17 were downregulated. The primary tissues of origin for these proteins were the colon and small intestine (p<1.6×10−6). Protein-protein interaction analysis demonstrated three predominant protein clusters (FIG. 1D) centered around: 1) innate immune response/cytokine production (false discovery rate [FDR]=8.76×10−20), 2) maintenance of gastrointestinal epithelium (FDR=0.002), and 3) angiogenesis and endothelial migration (FDR=0.00042).

Upregulated biomarkers may display greater than about 1× normalized protein expression, for example greater than about 1.5×, 2×, 3×, 4×, 5×, 6×, 7×, 8×, 9×, 10×, and less than about 15×, 11×, 10×, 9×, 8×, 7×, 6×, 5×, 4×, 2×, or 2×, whereas downregulated biomarkers may display expression that greater than about 40% decrease in normalized protein expression, for example greater than 40%, 45%, 50%, 55%, 60%, 65%, 70%, 75%, 80%, 85%, 90%, or 95% decrease, and less than about 99%, 95%, 90%, 85%, 80%, 75%, 70%, 65%, 60%, 55%, 50%, or 45% decreased expression.

The biomarker may be selected from one or more of LRRN1, VASH1, NELL2,DNAJB8, IGFBP1, PTPRN2, AFP, CD74, BST2, CD99, KIR3DL1, RBP2, CES2, NCS1,C4BPB, RAB6A, CCL4, FABP2, FUCA1, REG3A, GGA1, ATXN10, MUC13, ODAM, ARID4B, CCL2, CLEC11A, ITGAM, INPP1, ICOSLG, LRP1, MMP12, GBP2, GZMB, EPO, CXCL9, LGALS4, VIM, GZMH, MFAP5, CD46, SSB, CTSS, JAM2, KIR2DL3, BGN, PDGFC, RAD23B, FAM3B, GALNT7, VAT1, HBEGF, MIA, TPT1, IL1R1, CD83, MLN, CD200, CLEC4D, CLEC4C, CXLC10, BTN2A1, TFF2, CTRC, EPHX2, CHEK2, MFAP3, DCN, LGALS1, THBD, CDH1, FCN2, IL6R, ILKAP, FOSB, MASP1, DRAXIN, ABHD14B, CD274, ADAM8, SCARF 2, RGMA, DSC2, AKT3, NTF4, TBLX1, LYPD8, LYAR, ITGB1BP1, GH2, EPHA2, LRRC25, ALPP, LAG3, KLK4, CD300LF, HBQ1, FURIN, FLT4, FGFR2, NECTIN4, P4HB, ACTN4, WAS, HLA-E, PARP1, NT5C3A, CASP2, OSM, HSD11B1, SLAMF7, IL32, CCL3, TRIM21, TRAF2, ALDH3A1, EDAR, KLRB1, DFFA, TGFB1, REG4, CXCL1, CEP43, IRAG2, TP53INP1, GLO1, GRK5, PDGFRA, SDC4, PRKAR1A, FADD, GDF2, FAM3C, CASP3, MB, PAM, SERPINE1, TGFBI, ITGB1, CD93, ATP6V1F, BAX, NDRG1, SIRT5, MDGA1, STAMBP, EREG, IMPA1, CRADD, TNR, NPM1, PRDX1, PSME1, LAYN, CDH15, VTA1, NSFL1C, MSR1, TFF1, CLEC14A, bin2, PARK7, CCT5, SFTPA2, CRACR2A, SUGT1, CHAC2, GNE, FMR1, CDC27, CEP20, TBC1D23, UBAC1, OMG, VPS37A, TMPRSS15, KIFBP, CD207, CNPY4, CALCOCO1, CPE, METAP2, PPY, CDC37, FGFBP1, FGF5, SMPDL3A, NPPB, AMY2B, GUSB, ICAM2, CELA3A, PROC, PAEP, and ASAH2. In one embodiment, the biomarker is selected from LRRN1, VASH1, NELL2, DNAJB8, IGFBP1, PTPRN2, AFP, CD74, BST2, CD99, KIR3DL1, RBP2, CES2, NCS1, C4BPB, RAB6A, CCL4, FABP2, FUCA1, REG3A, GGA1, ATXN10, MUC13, ODAM, ARID4B, CCL2, CLEC11A, ITGAM, INPP1, ICOSLG, LRP1, and MMP12, for example LRRN1, VASH1, NELL2, DNAJB8, IGFBP1, PTPRN2, AFP, CD74, BST2, CD99, KIR3DL1, RBP2, CES2, NCS1, and C4BPB. In many embodiments, the biomarker is FABP2.

The biomarker may be upregulated or downregulated relative to normalized protein expression. The upregulated biomarker maybe one or more of LRRN1, VASH1, NELL2, DNAJB8, IGFBP1, PTPRN2, AFP, CD74, BST2, CD99, KIR3DL1, RBP2, CES2, NCS1, RAB6A, CCL4, FABP2, REG3A, GGA1, ATXN10, MUC13, ODAM, ARID4B, CCL2, CLEC11A, ITGAM, INPP1, ICOSLG, LRP1, MMP12, GBP2, GZMB, EPO, CXCL9, LGALS4, VIM, GZMH, CD46, SSB, CTSS, JAM2, KIR2DL3, BGN, RAD23B, GALNT7, VAT1, HBEGF, MIA, TPT1, IL1R1, CD83, MLN, CD200, CLEC4D, CLEC4C, CXLC10, BTN2A1, TFF2, EPHX2, CHEK2, MFAP3, DCN, LGALS1, THBD, CDH1, FCN2, ILKAP, FOSB, MASP1, DRAXIN, ABHD14B, CD274, ADAM8, SCARF 2, RGMA, AKT3, TBLX1, LYPD8, LYAR, ITGB1BP1, GH2, EPHA2, LRRC25, ALPP, LAG3, KLK4, CD300LF, HBQ1, FURIN, FLT4, FGFR2, NECTIN4, P4HB, ACTN4, WAS, HLA-E, PARP1, NT5C3A, CASP2, OSM, HSD11B1, SLAMF7, IL32, CCL3, TRIM21, TRAF2, ALDH3A1, EDAR, KLRB1, DFFA, TGFB1, REG4, CXCL1, CEP43, IRAG2, TP53INP1, GLO1, GRK5, PDGFRA, SDC4, PRKAR1A, FADD, GDF2, FAM3C, CASP3, MB, PAM, SERPINE1, TGFB1, ITGB1, CD93, ATP6V1F, BAX, NDRG1, SIRT5, MDGA1, STAMBP, EREG, IMPA1, CRADD, TNR, NPM1, PRDX1, PSME1, LAYN, CDH15, VTA1, NSFL1C, MSR1, TFF1, CLEC14A, bin2, PARK7, CCT5, SFTPA2, CRACR2A, SUGT1, CHAC2, GNE, FMR1, CDC27, CEP20, TBC1D23, UBAC1, OMG, VPS37A, TMPRSS15, KIFBP, CD207, CNPY4, CALCOCO1, CPE, METAP2, PPY, CDC37, FGFBP1, FGF5, NPPB, AMY2B, ICAM2, PROC, PAEP, and ASAH2. The downregulated biomarker maybe one or more of C4BPB, FUCA1, MFAP5, PDGFC, HMOX1, LBP, ILIRL2, TPP1, FAM3B, CTRC, IL6R, DSC2, NTF4, SMPDL3A, GUSB, and CELA3A. The biomarker expression may indicated susceptibility or risk of developing NEC in a subject with and/or having been treated for CHD. In many embodiments, the level of risk may correlate with the level of normalized expression.

This pilot analysis provides the first systematic exploration of the widespread changes in circulating proteins associated with cardiac NEC. IFABP shows promise as a diagnostic biomarker in this population, along with its potential use in treatment monitoring and prognosis. In addition, this study provides preliminary biomarker evidence for systemic immune upregulation, gastrointestinal epithelial disruption, angiogenesis, and endothelial migration in neonates who develop cardiac NEC. Studies may focus on 1) evaluating IFABP as a diagnostic/therapeutic monitoring biomarker in a large validation cohort, 2) assessing if a panel of our top performing biomarkers can improve diagnostic accuracy over IFABP alone, and 3) determine if NEC phenotypes (e.g., ischemic vs allergic) demonstrate distinct proteomic signatures that can be used to personalize therapy and inform mechanistic studies.

Expanding the size of this cohort and the frequency of longitudinal sampling, may be useful in developing a clinical biomarker panel for cardiac NEC and advancing our understanding of the pathogenesis of this complex disease.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

Affinity may refer to the avidity of one molecule for another—for example an antibody for a target antigen. In some embodiments, affinity can be presented as a relative binding—e.g. competition of two antibodies for the same target molecule.

“Amino acid identity,” “residue identity,” “identity,” and the like, as used herein refers to the structure of the functional group (R group) on the poly peptide backbone at a given position. Naturally occurring amino acid identities are (name/3-letter code/one-letter code): alanine/ala/A; arginine/arg/R; asparagine/asn/N; aspartic acid/asp/D; cysteine/cys/C; glutamine/gln/Q; glutamic acid/glu/E; glycine/gly/G; histidine/his/H; isoleucine/ile/I; leucine/leu/L; lysine/lys/K; methionine/met/M; phenylalanine/phe/F; proline/pro/P; serine/ser/S; threonine/thr/T; tryptophan/trp/W; tyrosine/tyr/Y; and valine/val/V.

An amino acid within a molecule may be substituted to create an engineered molecule. The amino acid (aa or a.a.) residue can be replaced by a residue having similar physiochemical characteristics, that is a ‘conservative substitution’—e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, for example based on size, charge, polarity, hydrophobicity, chain rigidity/orientation, etc., are well known in the art of protein engineering. Polypeptides comprising conservative amino acid substitutions can be tested in any one of the assays described herein to confirm that a desired activity, e.g. binding, specificity, and/or function of a native or reference polypeptide is achieved.

While conservative substitutions within a protein, i.e. buried or non-solvent accessible residues/positions, may in some cases alter the structure of the protein or affect folding of the protein, conservative substitutions at or near the protein's surface, i.e. exposed or solvent accessible residues/positions may cause little or no discernable change to the protein's structure and/or function, unless the altered surface protein is necessary for an interaction with another molecule, peptide, or protein. It is well within the abilities of the skilled artisan to alter the disclosed protein sequences by introducing conservative substitutions at up to 20% of the residues/positions without disrupting or changing the protein's structure and/or function.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: leucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and understood by those of skill in the art.

Within the definition of “antibody” according to the invention are full-length antibodies, antibody fragments, and antigen binding proteins. Also included are camelid antibodies and other immunoglobulins generated by biotechnological or protein engineering methods or processes. Full-length antibodies may be for example monoclonal, recombinant, chimeric, deimmunized, humanized and human antibodies, as well as antibodies from other species such as mouse, hamster, rabbit, rat, goat, or non-human primates.

Antibody fragments include antigen-binding portions of the antibody including, inter alia, Fab, Fab′, F(ab′)2, Fv, domain antibody (dAb), complementarity determining region (CDR) fragments, CDR-grafted antibodies, single-chain antibodies (scFv), single chain antibody fragments, chimeric antibodies, diabodies, triabodies, tetrabodies, minibody, linear antibody; chelating recombinant antibody, a tribody or bibody, an intrabody, a nanobody, a small modular immunopharmaceutical (SMIP), an antigen-binding-domain immunoglobulin fusion protein, single domain antibodies (including camelized antibody), a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity.

The term “antibody” may also be used to refer to a molecule in which the structure and/or function is/are based on the structure and/or function of an antibody, e.g., of a full-length immunoglobulin molecule. An antibody construct hence immunospecifically binds to its target or antigen, and/or it comprises domains which are derived from or which are the heavy chain variable region (VH) and/or the light chain variable region (VL) of an antibody. Furthermore, an antibody construct according to the invention comprises the minimum structural requirements of an antibody which allow for immunospecific target binding. This minimum requirement may e.g. be defined by the presence of at least three light chain CDRs (i.e. CDR1, CDR2 and CDR3 of the VL region) and/or three heavy chain CDRs (i.e. CDR1, CDR2 and CDR3 of the VH region), preferably of all six CDRs.

“Antibody” of the present invention may also comprise fragments of full-length antibodies, such as VH, VHH, VL, (s) dAb, Fv, light chain (VL-CL), Fd (VH-CH1), heavy chain, Fab, Fab′, F(ab′).sub.2 or “r IgG” (“half antibody” consisting of a heavy chain and a light chain). Antibody constructs according to the invention may also comprise modified fragments of antibodies, also called antibody variants or antibody derivatives. Examples include, but are not limited to, scFv, di-scFv or bi(s)-scFv, scFv-Fc, scFv-zipper, scFab, Fab.sub.2, Fab.sub.3, diabodies, single chain diabodies, tandem diabodies (Tandab's), tandem di-scFv, tandem tri-scFv, “minibodies” exemplified by a structure which is as follows: (VH-VL-CH3).sub.2, (scFv-CH3).sub.2, ((scFv).sub.2-CH3+CH3), ((scFv).sub.2-CH3) or (scFv-CH3-scFv).sub.2, multibodies such as triabodies or tetrabodies, and single domain antibodies such as nanobodies or single variable domain antibodies comprising merely one variable region, which might be VHH, VH or VL, that specifically binds to an antigen or target independently of other variable regions or domains. Further possible formats of the antibody constructs according to the invention are cross bodies, maxi bodies, hetero Fc constructs, mono Fc constructs and scFc constructs.

The term “antibody” or “immunoglobulin” refers to a tetrameric glycoprotein that consists of two heavy chains and two light chains, cach comprising a variable region and a constant region. “Heavy Chains” and “Light Chains” refer to substantially full length canonical immunoglobulin light and heavy chains (see e.g., Immunobiology, 5th Edition (Janeway and Travers et al., Eds., 2001). Antigen-binding portions may be produced by recombinant DNA techniques or by enzymatic or chemical cleavage of intact antibodies. The term “antibody” includes monoclonal antibodies, polyclonal antibodies, chimeric antibodies, human antibodies, and humanized antibodies.

Antibody variants include antibody fragments and anti-body like proteins with changes to structure of canonical tetrameric antibodies. Typically antibody variants include V regions with a change to the constant regions, or, alternatively, adding V regions to constant regions, optionally in a non-canonical way. Examples include multispecific antibodies (e.g., bispecific antibodies with extra V regions), antibody fragments that can bind an antigen (e.g., Fab′, F′(ab)2, Fv, single chain antibodies, diabodies), biparatopic and recombinant peptides comprising the forgoing as long as they exhibit the desired biological activity.

“Antigen” refers to a compound, composition, substance, protein, peptide, nucleic acid, nucleo-peptide, etc., whether native, modified, or synthetic, that can stimulate the production of antibodies or a T-cell response in an animal, including compositions that are injected or absorbed into an animal or modified by an animal. As used herein, an antigen may be defined by its ability to bind to or within a binding site of a native or engineered molecule. In some aspects, an antigen may react with one or more products of specific humoral or cellular immune system. The term “antigen” includes all related antigenic epitopes and antigenic determinants.

A “biomarker” is a characteristic that is objectively measured and evaluated as an indicator of normal biological processes, pathogenic processes, or pharmacological responses to a therapeutic intervention. Biomarkers may be of several types: predictive, prognostic, or pharmacodynamics (PD). Predictive biomarkers predict which patients are likely to respond or benefit from a particular therapy. Prognostic biomarkers predict the likely course of the patient's disease and may guide treatment. Pharmacodynamic biomarkers confirm drug activity, and enables optimization of dose and administration schedule.

“Diagnose” or “diagnostic” refers identifying and/or detecting the presence or absence of or nature of a disease or disorder. Such detection methods can be used, for example, for early diagnosis of the condition, to determine whether a subject is predisposed to a disease or disorder, to monitor the progress of the disease or disorder or the progress of treatment protocols, to assess the severity of the disease or disorder, to forecast the an outcome of a disease or disorder and/or prospects of recovery, or to aid in the determination of a suitable treatment for a subject. Disclosed herein are compositions and methods useful in detecting and/or diagnosing various diseases, disorders, and conditions, which may be characterized by one or more symptoms associated with, for example necrotizing enterocolitis (NEC), and/or congenital heart discase (CHD), or any of the diseases and conditions disclosed within. In many cases, detecting may involve combining a biomarker with a detecting molecule having affinity for the biomarker. The molecule may then be detected, isolated, identified or analyzed by various methods known to those of skill in the art. In one embodiment, without wishing to be restricted to one method, the detection involves the molecule comprising one or more nucleic acid sequences that may be amplified for detection by nucleic acid detecting methods.

“Expression” as used herein, refers to cellular processes involved in producing, displaying (e.g., on or at a cell's surface/outer membrane), or secreting RNA and proteins including where applicable, but not limited to, for example, transcription, transcript processing, translation and protein folding, modification and processing. Expression can refer to the transcription and stable accumulation of sense (e.g., mRNA) or antisense RNA derived from a nucleic acid fragment or fragments and/or to the translation of mRNA into a polypeptide.

Similarity between amino acid or peptide sequences is expressed in terms of the homology of two sequences, otherwise referred to as sequence identity. Sequence identity is frequently measured in terms of percentage identity (percentage of identical residues for peptides or bases for nucleic acids; or similarity or homology); the higher the percentage, the more similar the two sequences are. Complete identity is 100% identical over a given sequence, for example 50, 100, 150, or 200 bases or residues. In many embodiments, the level of identity to the biomarker protein sequence maybe greater than about 80%, 85%, 90%, 95%, 96%, 97%, 98%, or 99%, and less than about 100%, 99%, 98%, 97%, 96%, 95%, 90%, and 85%.

The term “mammal” includes, but is not limited to, humans, mice, rats, guinea pigs, monkeys, dogs, cats, horses, cows, pigs and sheep.

The terms “modulate”, “modulation” and the like refer to the ability of a compound to increase or decrease the function, or activity of an organism, cell, protein, peptide, gene, biomarker, etc (“target”). “Modulation”, in its various forms, is intended to encompass inhibition, antagonism, partial antagonism, activation, agonism and/or partial agonism of the activity associated with the target. Inhibitors compounds may bind to, partially or totally block stimulation, decrease, prevent, delay activation, inactivate, desensitize, or down regulate signal transduction. The ability of a compound to modulate a target's activity can be demonstrated in various ways, such as nucleic acid quantitation (northern analysis), an enzymatic assay or a cell-based assay.

“Nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA, cDNA, or vector DNA. Suitable RNA can include, e.g., mRNA.

DNA (deoxyribonucleic acid) and RNA (ribonucleic acid) refer to nucleic acid molecules having a backbone of sugar moieties which are deoxyribosyl and ribosyl moieties respectively. The sugar moieties may be linked to bases which are the 4 natural bases (adenine (A), guanine (G), cytosine (C), thymine (T), and uracil (U)). In RNA, the bases are A, G, C, and U. The sugar moieties may also be linked to unnatural bases such as inosine, xanthosine, 7-methylguanosine, dihydrouridine and 5-methylcytidine. Other unnatural bases? Natural phosphodiester linkages between sugar (deoxyribosyl/ribosyl) moieties may optionally be replaced with phosphorothioates linkages.

Nucleic acids of the present disclosure may be RNA, in particular microRNA or miRNA. miRNA may refer to small, non-coding, single-stranded RNA molecules (containing from about 18 to about 28 nucleotides), that may function in post-transcriptional regulation of gene expression. In many cases, miRNAs function via base-pairing with complementary sequences within target mRNA molecules—i.e. mRNA transcribed from a target gene.

A “package insert” is a leaflet that, by order of the Food and Drug Administration (FDA) or other Regulatory Authority, must be placed inside the package of every prescription drug. The leaflet generally includes the trademark for the drug, its generic name, and its mechanism of action; states its indications, contraindications, warnings, precautions, adverse effects, and dosage forms; and includes instructions for the recommended dose, time, and route of administration.

A “patient” or “subject” includes a mammal or animal, such as a human, cow, horse, sheep, lamb, pig, chicken, turkey, quail, cat, dog, mouse, rat, rabbit, or guinea pig. The animal can be a mammal such as a non-primate or a primate (e.g., monkey and human). In one embodiment, a patient is a human, such as a human infant, child, adolescent, or adult of any or indeterminant sex. In most embodiments disclosed herein the subject or patient is a newborn child.

“Prevention” as used herein means the avoidance of the occurrence or of the re-occurrence of a disease, disorder, or condition as specified herein, by the administration of a composition, compound, treatment, or therapy according to the present disclosure to a subject in need thereof.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

The term “sample” or “biological sample” refers to a specimen obtained from a subject for use in the present methods, and includes urine, whole blood, plasma, scrum, saliva, sputum, tissue biopsies, cerebrospinal fluid, peripheral blood mononuclear cells with in vitro stimulation, peripheral blood mononuclear cells without in vitro stimulation, gut lymphoid tissues with in vitro stimulation, gut lymphoid tissues without in vitro stimulation, gut lavage, bronchioalveolar lavage, nasal lavage, and induced sputum.

The term “specifically binds” is “antigen specific”, is “specific for”, “selective binding agent”, “specific binding agent”, “antigen target” or is “immunoreactive” with an antigen refers to an antibody or polypeptide that binds a target antigen with greater affinity than other antigens of similar sequence. In some embodiments with another but can cross-react with an ortholog of a closely related species, e.g. an antibody may bind a human protein and also bind a closely related primate protein.

“Subject in need,” “patient” or those “in need of treatment” include those already with existing disease (i.e. CHD, for example), and at risk of developing another, for example without limitation, NEC, as well as those at risk of or susceptible to these diseases. The terms also include human and other mammalian subjects that receive either prophylactic or therapeutic treatments as disclosed herein.

The terms “treat,” “treating,” and “treatment” refer to eliminating, reducing, suppressing, or ameliorating, either temporarily or permanently, either partially or completely, a clinical symptom, manifestation or progression of an event, disease or condition associated with immune disorders and diseases described herein. As is recognized in the pertinent field, methods and compositions employed as therapies may reduce the severity of a given disease state but need not abolish every manifestation of the disease to be regarded as useful. Similarly, a prophylactically administered treatment need not be completely effective in preventing the onset of a condition to constitute a viable prophylactic method or agent. Simply reducing the impact of a disease (for example, as disclosed herein, CHD and/or NEC, etc. and/or reducing the number or severity of associated symptoms, or by increasing the effectiveness of another treatment, or by producing another beneficial effect), or reducing the likelihood that the disease will occur or worsen in a subject, is sufficient. One embodiment of the present disclosure is directed to a method for determining the efficacy of treatment comprising administering to a patient therapeutic treatment in an amount, duration, and repetition sufficient to induce a sustained improvement over pre-existing conditions, or a baseline indicator that reflects the severity of the particular disorder.

Further exemplary embodiments are disclosed below in the Examples and the disclosure are provided and described for all non-limiting purposes.

From the foregoing it will be appreciated that, although specific embodiments of the disclosure have been described herein for purposes of illustration, various modifications may be made without deviating from the spirit and scope of the disclosure. Accordingly, the disclosure is not limited except as by the appended claims.

### EXAMPLES

### Example 1—Overcoming the Significant Limitations of Single Biomarker Research in Neonates With CHD.

Applicants are first to apply proteomics approaches to evaluate postoperative morbidity following neonatal CHD surgery. Applicant's study design allows for unbiased identification of previously unidentified candidate biomarkers, a key innovation in the present approach lies in use of proteomic analysis to also support hypothesis-driven, targeted biomarker research and mechanistic exploration. First, Applicants use proteomics as a highly efficient method to validate previously identified candidate biomarkers across multiple postoperative morbidities. Second, this approach allows for novel evaluations of how targeted biomarker evidence of injury or other pathology is associated with both predicted and unpredicted changes in other protein systems. For example, the present approach allows validation of markers of postoperative intestinal injury, and determining if patients with biomarker evidence of intestinal injury develop a systemic cytokine response and if the makeup of this response is associated with severity of illness or subsequent recovery, which would not be possible with a single biomarker or biomarker panel design and represents a unique opportunity inherent to the proposed approach.

The innovations disclosed herein are made feasible by a novel application of OLINK proteomics technology (Uppsala, Sweden) to the present high-risk population. While the use of this proximity extension assay for proteomic profiling is established, it has unrealized potential to advance neonatal CHD research. The key to this potential is the extremely low sample volume required for this assay (<20 μL), making longitudinal sampling in this vulnerable population viable for the first time. In fact, the required volume is so small that it can be analyzed using the tiny amount of plasma remaining following clinical chemistry tests, obviating the need for additional research blood draws. Applicants leverage testing alongside routine clinical chemistries to perform frequent proteomic monitoring, allowing detection of subclinical changes in protein biomarkers prior to the onset of overt clinical events. Furthermore, this strategy is used to follow the proteomic shifts through acute clinical events and into recovery.

Traditional approaches to biomarker research are inherently inefficient, requiring recruitment of a new patient cohort (usually over several years) to answer a small number of related questions. Biobanking represents one option to improve the efficiency of biomarker studies. Biobanking, however, suffers from significant shortcomings including the substantial storage cost/logistics, sample degradation over time, difficulty tracking sample availability and relevant subpopulations, and difficulty sharing the samples with the international community. In this study, Applicant's use an innovative strategy of building a publicly available data bank for the free international study of-omics and clinical data. This strategy converts biologic samples into a comprehensive data bank of proteins and metabolites along with deidentified clinical data, efficiently targeting multiple comorbidities in a single study and eliminating the need for recruitment of multiple cohorts or costly biobanking.

### Example 2—Determining Proteomic Phenotype and Dysregulated Protein Systems in Neonates Who Develop Key Clinical Cardiovascular Complications After CHD Surgery Including Mace, Cardiogenic Shock, and Vasoplegic Shock

Applicants hypothesized that postoperative injury following neonatal CHD surgery results in disruption of the circulating proteome during injury, repair, and longitudinal growth. Serial measurements of the postoperative proteome are useful in validating candidate biomarkers of MACE, NEC, and neurologic injury, defining additional novel biomarkers, and informing potential mechanisms through network approaches to systems biology analysis.

Disclosed herein are early postoperative proteomic signatures, differentially expressed proteins, and dysregulated protein systems that a) precede MACE and b) are associated with increased postoperative cardiovascular support requirements after neonatal CHD surgery.

Applicants validate acute changes in the circulating proteome following neonatal CHD surgery using the OLINK 3072 platform, determine specific proteomic phenotype and differentially dysregulated protein networks of subjects with and without subsequent MACE (cardiac arrest, extracorporeal membrane oxygenation [ECMO], or death), as well as candidate protein predictors of subsequent MACE.

Here, Applicants hypothesized that i) Neonatal CHD surgery results in widespread changes in the circulating proteome, including 1) upregulation of cytokine production, metabolism (aerobic glycolysis, amino acid catabolismaD production), and molecular signaling pathways (cAMP catabolismsMAD signaling, growth factor signaling) and 2) downregulation of lipoprotein/chylomicron formation, potassium/sodium transport, extracellular matrix (ECM) regulation, JAK-STAT signaling, interleukin (IL)-2/T-cell signaling, and astrocyte/glial cell development; ii) Neonates developing postoperative MACE demonstrate evidence of greater postoperative metabolic, inflammatory, electrolyte transport, ECM, and signal transduction dysregulation; and iii) Dysregulated proteins at 2 hrs postoperatively have additive predictive value to clinical physiologic measurements for the identification of neonates at high risk for subsequent MACE.

Proteomics of postoperative cardiovascular support and cardiovascular phenotype. Here, Applicants determine the pre- and postoperative proteomic phenotypes/protein networks associated with increased postoperative cardiovascular support requirements (vasoactive inotropic score [VIS] at 24 and 48 hrs postoperatively), and combine the proteomic data with dense physiologic data to determine the molecular phenotypes of postoperative vasoplegic versus cardiogenic shock.

Applicants hypothesized that neonates with higher VIS scores at 24 and 48 hrs postoperatively demonstrate greater proteomic evidence of altered cAMP, MAPK/RAS, and JAK-STAT signaling, increased leukocyte/complement activation, mitochondrial disorganization, and aortic pathology pathways at 2 and 24 hrs postoperatively, and neonates with a clinical cardiogenic shock phenotype (poor cardiac function, low pulse pressure, high arteriovenous oxygen difference [AVDO2]) are distinguishable from neonates with a vasoplegic phenotype (good cardiac function, wide pulse pressure, low AVDO2) based on their proteomic profile and dysregulated protein networks.

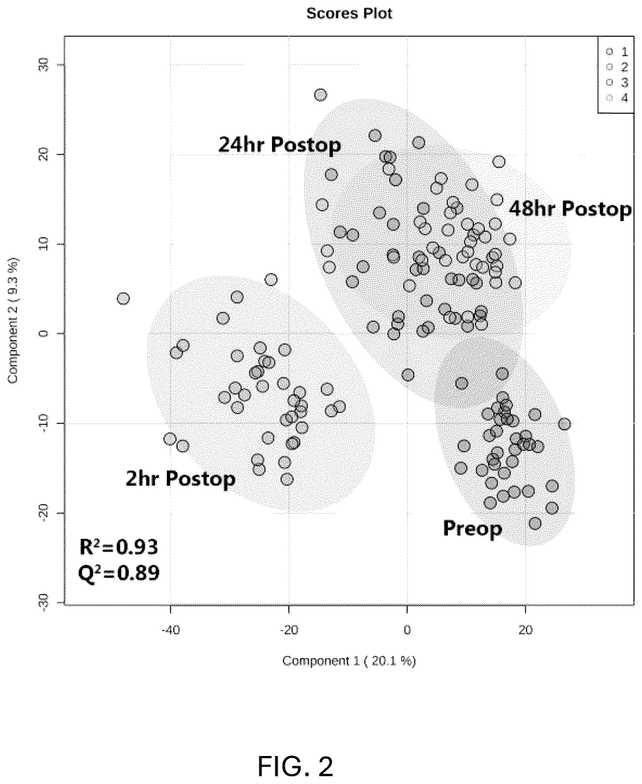

For this study, Applicants analyzed the circulating proteome in 38 neonates and young infants undergoing CHD surgery with CPB through 48 hrs postoperatively. Of the 1512 proteins measured, 1268 (84%) were significantly dysregulated postoperatively compared to baseline after correction for multiple comparisons. The global proteome diverged most from baseline at 2 hrs postoperatively but remained altered at both 24 and 48 hrs (FIG. 2). The most common patterns of change for individual proteins were a 2 hr peak (498 proteins) or 2 hr nadir (274 proteins) followed by either persistent depression (143 proteins) or persistent elevation (80 proteins). Following cluster and protein network analysis, evidence for overrepresentation of multiple protein systems was identified. These systems included increased levels of proteins involved in glycolysis (false discovery rate [FDR]=2×10−16), purine and amino acid catabolism (FDR=4×10−7), NAD production (FDR=0.04), BMP/SMAD/TGF signaling (FDR=0.0003), MAPK/Ras signaling (FDR=5.6×10−21), myocardial damage (FDR=2×10−13), growth factor/ECM regulation (FDR=4.6×10−31), and cytokine signaling (FDR=1.5×10−36). In contrast Applicants identified decreased levels of proteins involved in IL-2 signaling/T-cell regulation (FDR=3.5×10−7), JAK/STAT signaling (FDR=6×10−11), lipid metabolism/chylomicron formation (FDR=5×10−8), PI3K/AKT signaling (FDR=7×10−7), and metal ion binding (FDR=0.01) early after surgery and persistent depression of proteins involved in glial cell/astrocyte development (FDR=0.0002), IL-10/TNF signaling (FDR=0.002), short chain fatty acid metabolism (FDR=0.03), and oxidative stress response (FDR=0.0008).

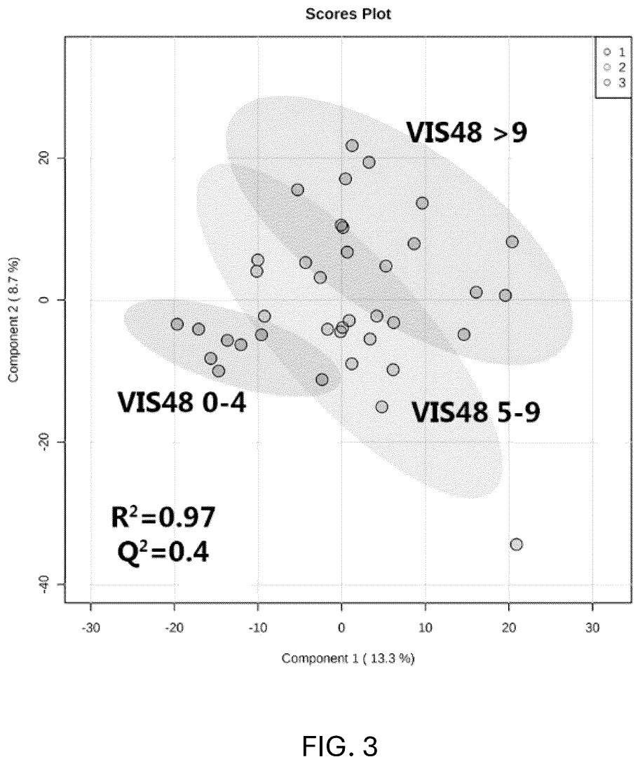

Next, Applicants evaluated if the circulating proteome at 2 hrs postoperatively could discriminate among subjects with higher versus lower vasoactive-inotropic support requirements at 48 hrs postoperatively (VIS48; FIG. 3). Despite the small cohort size, the global proteome at 2 hrs strongly discriminated groups by VIS48 (R2=0.97) with moderate reproducibility on cross- validation (Q2=0.4). Key protein systems identified on network analysis included JAK/STAT signaling (FDR=7.6×10−8), cell matrix adhesion/angiogenesis (FDR=3.7×10−7), aortic development (FDR=2×10−7), complement activation (FDR=1.7×10−6), and apoptosis modulation (FDR=8.9×10−6).

This study identifies promising candidate protein predictors of MACE, which may be incorporated into a novel targeted protein panel (with/without clinical physiologic variables) to form a novel predictive tool for subsequent clinical validation in a multicenter clinical trial. Additionally, as a result of identifying candidate mechanisms underlying MACE, cardiogenic shock, and vasoplegic shock in this population, Applicants also identify dysregulated protein networks in this cohort allowing subsequent targeted studies focused on specific metabolic, cellular, and signaling pathways. Based on identified protein pathways, a robust series of investigations may be pursued-ranging from immediate opportunities for biomarker directed clinical trials (e.g., metabolic and micro/macronutrient studies, circulating PDE3a level guidance of milrinone therapy), to mechanistic trials of existing signaling modulation therapies in our large animal CPB model (e.g., JAK-STAT modulators), to early-stage mechanistic studies (e.g., ECM regulation, postnatal NOTCH signaling).

Experiments for this aspect were performed as briefly described. A prospective cohort study design is followed in CHCO cardiovascular operating room and cardiac ICU. Patients are selected as follows—Neonate (<29 days of age) undergoing cardiothoracic surgery with CPB were included, while a weight at surgery of <2 kg (increased risk of anemia with repeat blood sampling) excluded the patient from the study. Primary clinical outcomes include MACE (cardiac arrest, ECMO, or death, in hospital or within 30 days, while primary testing includes analysis of proteomic profile. Secondary clinical outcomes include a) VIS at 2, 24, and 48 hrs postoperatively, b) primary clinical cardiovascular phenotype (based on continuous arterial blood pressure, head/flank near infrared spectrometry, and echocardiographic ventricular function), c) duration of ICU and hospital length of stay (LOS), d) duration of mechanical ventilation, e) development of acute kidney injury.

Plasma samples for proteomic profiling are obtained preoperatively (after induction of anesthesia) and at 2 (+/−1), 24 (+/−4), and 48 (+/−8) hours postoperatively. Samples are processed immediately and stored at −80° C. for batch analysis. All Aim 1 samples are obtained as research draws to standardize timing and minimize missing data during the acute postoperative period.

Samples are analyzed using the OLINK Explore 3072 panel, which measures a library of 3072 proteins using <20 μL of plasma. The list of measured proteins can be found at www.olink.com. OLINK uses a proximity extension assay to measure the relative abundance of the protein targets. Briefly, for each protein, oligonucleotide-labeled antibody pairs bind to specific epitopes on the protein surface, and complementary oligonucleotide sequences give rise to DNA reporter sequences, which are then quantified using real-time PCR.

Subjects are enrolled under an existing, IRB approved R01-funded cohort study that targets the metabolomic response of infants (≤120 days of age) to cardiac surgery and postoperative cardiac failure. Consent includes sample banking for the proteomic analyses in this proposal. Enrollment is about 150 neonates. In the first 18 months of recruitment in the parent study, Applicants enrolled 63 neonates and banked samples for proteomic analysis (3-4 subjects/month), and expect to complete enrollment of 150 total subjects.

Applicants' disclosed method overcomes drawbacks of typical ELISA or multiplex assays, which are both limited in the scope of questions that can be answered (single biomarkers or small panels of related biomarkers) and require large enough sample volumes (typically 30-100 μL) that they present a risk of anemia in these small, fragile patients if sampling occurs too frequently. Applicants' longitudinal sampling uses minute plasma samples (<20 μL) to measure over 3000 proteins. This technique allows efficient measuring of multiple related and unrelated biomarkers using plasma volumes easily available from residual clinical samples, reducing the need for research-specific blood draws.

Power calculations are performed based on a preliminary data set of 38 infants where the abundance of 1,512 circulating proteins was measured. Due to the small number of subjects experiencing MACE in this pilot group, the power analysis was performed using Vasoactive-Inotropic Score at 48 h (VIS48) as the outcome. A power test was performed for each protein to determine the number of subjects needed to achieve the desired power to detect differences between proteins in the three VIS48 tiers (see FIG. 3). Power.anova.test was used in the R statistical programming language, where highly stringent parameters were set: power of 0.9, significance level of 0.05/1512 (i.e. Bonferroni correction), and taking the within-group variance to be the maximum found in any of the three tiers. From this analysis Applicants determined how many subjects per group are needed to detect a certain number of significant proteins: 25 subjects/group to detect 10 significant proteins and 51 subjects/group for 100 proteins. Thus, with a sample size of 150 subjects (˜50 per VIS48 tier) a power of 0.9 detects the top 100 proteins differing by VIS48 tier.

All analyses in all Aims include both analysis of the full cohort and stratification or multivariable modeling by sex to evaluate the effect of sex as a biologic variable. To capture the broad changes of the proteome unsupervised and supervised dimension reduction analysis was performed (principal component analysis and PLS-DA respectively), assessing how well the global proteome differentiates among clinical groups of interest (e.g., time points, MACE, VIS levels, cardiovascular shock phenotypes). To evaluate individual proteins, the distribution of the data is assessed and log transformation performed, as needed, to approximate a normal distribution. Next t-tests, ANOVA, or non-parametric equivalents (as indicated by the specific comparisons and data distributions) are performed separately for each protein to detect changes between timepoints or groups, using an FDR p-value adjustment. For analyses with multiple groups, the Tukey's post-hoc test is used to measure specific differences between groups. Once a list of significant proteins is obtained for a specific research question, a proteome-wide systems biology analysis is performed. First, since the proteomics assay is incomplete and may be noisy, network-based techniques are used to computationally predict other proteins related to the original set. Then this is overlaid on top of a proteome-scale network and clustered. Proteins belonging to each cluster are input in the R-package clusterprofiler to perform over-representation analysis of Gene Ontology biological processes and pathways from KEGG and REACTOME.

### Example 3—Leveraging of Longitudinal Proteomic Analysis to 1) Perform a Detailed Analysis of the Protein Networks Activated Before and During Postoperative NEC, 2) Validate iFABP as a Biomarker of Postoperative NEC With and Without Additional Novel Protein Biomarkers, and 3) Determine if Food Protein Intolerance Colitis Can Be Differentiated From NEC Based on the Circulating Proteomic Profile

Applicants disclose determining changes in circulating proteins and protein systems associated with development of and recovery from NEC after neonatal CHD surgery, including targeted validation of intestinal fatty acid binding protein (iFABP) as a diagnostic and prognostic biomarker. Applicants describe using the OLINK 3072 platform, performing serial postoperative measurement of the circulating proteome to determine the changes in the global proteome and protein networks/biologic systems preceding NEC, at the time of acute NEC presentation, and during treatment/recovery from NEC. In this regard, Applicants hypothesized that subjects with NEC demonstrate broad changes to the circulating proteome with alteration of multiple molecular pathways involving inflammation, maintenance of gastrointestinal epithelium, and angiogenesis/endothelial migration, compared to subjects without NEC. These changes are hypothesized to precede overt clinical NEC, peak at onset of NEC, and shift to a distinct recovery phase during therapy. The subset of NEC subjects who clinically respond to therapy with hypoallergenic formula demonstrate a distinct proteomic phenotype compared to NEC subjects without an allergic clinical phenotype.

Applicants also disclose performing targeted serial postoperative iFABP measurements to 1) determine the extent and duration of subacute intestinal injury prior to development of clinical NEC, 2) assess the dose-response relationship between iFABP levels and NEC severity, and 3) evaluate the response of iFABP levels to therapy. Determine the predictive value of iFABP alone and in combination with novel protein biomarkers for the detection of NEC prior to the onset of clinical symptoms. In this regard, Applicants hypothesized that circulating iFABP levels increase prior to the development of clinical NEC, higher levels are associated with more severe disease, and levels decrease in response to successful therapy. Moreover, that limited protein biomarker panels including iFABP are useful to detect patients at risk for subsequent NEC with high sensitivity/specificity prior to the onset of clinical symptoms.

NEC is an important contributor to morbidity and mortality following neonatal CHD surgery: CHD is the most common cause of NEC in term infants, with an incidence up to 100 times higher than infants without CHD. NEC in CHD patients most commonly presents with hematochezia, followed by pneumatosis on abdominal radiograph, which can proceed to shock in some cases. NEC develops in 3-30% of infants with CHD depending on the specific population evaluated, with the highest incidence in neonates undergoing staged palliation for SVHD. NEC is strongly associated with mortality (25-50% unadjusted mortality rate), increased length of stay (LOS), and increased hospital charges following CHD surgery. Furthermore, NEC treatment is not benign, with frequent exposure to broad spectrum antibiotics (42% Bell Stage 1, 94% Stage 2/3) and disruption of enteral nutrition (withholding of enteral nutrition in 89% of Bell Stage 1 and 100% of Stage 2/3 cases). Neonates who develop NEC often require g-tube placement (39% Bell Stage 1, 63% Stage 2/3) suggesting that chronic intestinal dysfunction is a common comorbidity with NEC. Despite the serious impact of NEC on patient outcomes, the mechanisms underlying NEC in infants with CHD are poorly understood, and no biomarkers are clinically available to aid in diagnosis, prognosis, or guidance of therapy.

Applicants have found that circulating iFABP is a novel biomarker of enterocyte injury, showing promise in studies of NEC in premature infants. IFABP/iFABP/FABP2 is a 15-kD cytosolic protein localized to mature enterocytes of the intestinal villi with low circulating levels. Circulating iFABP is increased at the onset of NEC symptoms in premature infants with NEC, and levels are frequently higher with more severe disease. Levels peak within 24-48 hrs of diagnosis and then decrease in response to therapy except in severe cases where levels remain elevated. These findings suggest that iFABP may be useful both for diagnosis and to monitor response to therapy in premature infants with NEC. Less is known about iFABP as a biomarker in children with CHD. Serum iFABP levels increase immediately following both adult and pediatric cardiac surgery, indicating measurable enterocyte injury during surgery. The clinical importance of enterocyte injury following CHD surgery, however, is less clear, as studies examining associations between iFABP levels and postoperative NEC are lacking.

Longitudinal proteomic testing provides an opportunity to simultaneously target iFABP as a biomarker of postoperative NEC and examine the systemic response to NEC: Recently, as described above, Applicants identified higher immediate postoperative circulating iFABP levels in infants who subsequently developed postoperative NEC. While these findings suggest that infants with early postoperative enterocyte injury are at risk for future NEC, it is not known if subacute intestinal stress can be monitored longitudinally via iFABP levels, how circulating levels of iFABP change around acute NEC episodes, if iFABP levels reflect disease severity, or if iFAB levels normalize in response to therapy.

NEC is a multisystem disease including prominent inflammatory and hemodynamic responses. It is unlikely that iFABP alone is sufficient to decipher this complex process. A systems biology approach is needed to measure the multitude of pathways activated during NEC and begin to unravel the mechanisms behind this disease. This approach is especially important given the growing appreciation that clinical NEC may not represent a single disease entity. Infants with CHD who present with hematochezia and pneumatosis and are diagnosed with NEC, but who respond to hydrolyzed formula are often seen, suggesting an allergic mechanism similar to the food protein intolerance enterocolitis described in premature neonates. The ability to distinguish NEC from food protein intolerance could minimize both antibiotic exposure and time without enteral nutrition. Thus, a proteomic profiling strategy offers promise not only for identifying diagnostic, prognostic, and mechanistic biomarkers but also for guiding specific therapeutic decisions.

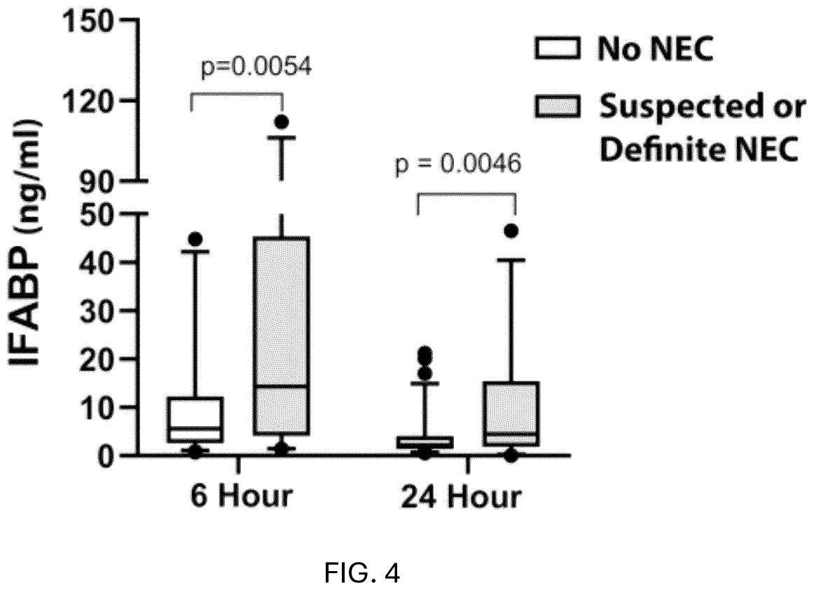

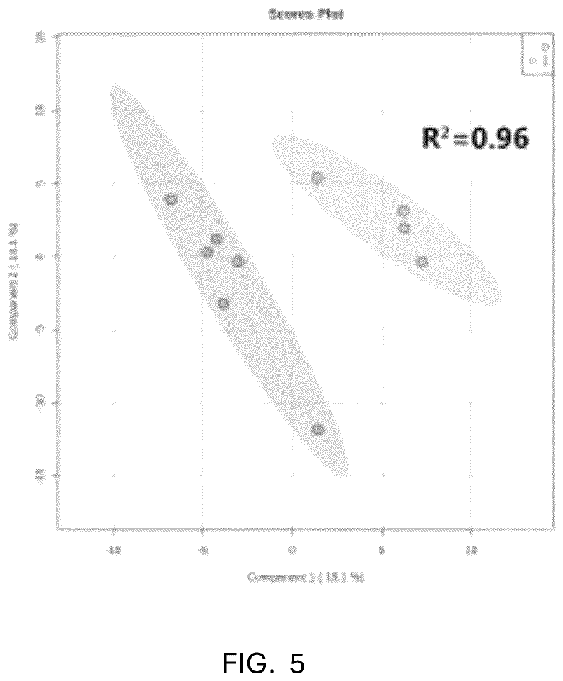

Applicants measured circulating iFABP in 102 infants undergoing CHD surgery to determine the association of early enterocyte injury with subsequent NEC. Approximately 25% of the cohort developed NEC. Subjects who later developed postoperative NEC demonstrated higher iFABP levels at both 6 and 24 hrs after surgery (FIG. 4). Six hour iFABP levels were independently associated with subsequent NEC on multivariable analysis (4% increase in odds of NEC per unit increase in iFABP; p=0.0015). Next, a pilot study was performed using the OLINK 1536 proteomics panel to determine changes in the circulating proteins at the time of clinical postoperative NEC, including targeted analysis of iFABP (FIG. 1A, 1B, and 1C). IFABP levels were significantly higher on NEC day I than in subjects who did not develop NEC (FIG. 1A) with levels decreasing by NEC day 5 except in one subject who did not recover and died 9 days after the NEC episode. Proteomic analysis identified 246 proteins that were significantly dysregulated (FIG. 5B: 229 upregulated, 17 downregulated); small intestine and colon were the most common tissues of origin (p<1.6×10−6). Protein-protein interaction analysis demonstrated three predominant protein clusters (FIG. 1C) centered around: 1) innate immune response/cytokine production (FDR=8.76×10−20), 2) maintenance of gastrointestinal epithelium (FDR=0.002), and 3) angiogenesis and endothelial migration (FDR=0.00042).

The disclosed experiments are able to validate iFABP and identify novel protein biomarkers of postop NEC to promote and support (1) early diagnosis, for example by allowing initiation of therapy prior to clinical deterioration, (2) accurate diagnosis through the limiting of enteral nutrition interruptions and unnecessary antibiotic exposure, and (3) mor accurate therapies through identification of distinct molecular phenotypes of NEC versus allergic colitis allowing early transition to hypoallergenic formula when indicated. Additionally, the disclosed experiment have longer term impacts, for example (1) the identification of the proteomic pathways most significantly altered during NEC to elucidate candidate mechanisms of injury/recovery and identify novel molecular targets for prevention and treatment of NEC after CHD surgery, (2) the development of precision medicine strategies to limit acute morbidity and length of hospitalization as well as chronic nutritional and growth effects caused by CHD-related NEC.

The disclosed experiments have the following study design and inclusion/exclusion criteria similar to those described above. Primary clinical measurements include episodes of NEC by modified Bell criteria, while secondary clinical outcomes measure duration of NEC, antibiotic administration/duration, days without enteral nutrition (NPO), total parenteral nutrition days, vasoactive/inotropic support, enteral nutrition type before and after NEC, home discharge enteral nutrition regimen. Primary biomarker outcomes include measurement of longitudinal plasma levels of iFABP during the immediate postoperative period and in-hospital convalescence, while secondary biomarker measurements include daily plasma levels of iFABP around acute NEC events; OLINK 3072 proteomic profile (longitudinal and surrounding NEC events).

For these experiments, blood sampling involves obtaining plasma samples of about 20 μL preoperatively, following induction of anesthesia, and at 2, 24, and 48 hrs postoperatively (acute postoperative) as above. Addition, weekly plasma samples are collected on postoperative day 7 until discharge, as well as daily for up to five days around the time of NEC. Sample collection is timed to coincide with either research draws during the acute postoperative time or at the time of clinical lab draws during convalescence, eliminating the need for additional central line access or venipuncture for this proposed study. The tiny sample volume needed for these assays is routinely available from residual clinical samples, so no additional blood volume is needed for these experiments. All blood samples were processed for plasma at the time of collection and stored at −80° C. for batch analysis. Samples are analyzed using the OLINK Explore 3072 panel as described above.

Power analysis involved power calculations performed based on preliminary data for iFABP (measured 24 hrs postoperatively) and NEC. The distribution of iFABP was skewed and non-normal, so power calculations were based on the Mann-Whitney U test with a two-sided type I error rate of 0.05. Applicants assumed that 25% of the study population experiences NEC (n=35-40) and 75% do not (n=110-115) and that the difference in median iFABP between the groups is at least as large as the difference observed in the preliminary data (2.2). A bootstrapping approach of Collings and Hamilton was employed with a median shift to conduct the power calculation. This indicates having 99.5% power to detect a difference in central tendency of iFABP levels in subjects with and without NEC.

A Mann-Whitney U test is used to assess whether circulating levels of iFABP taken 1-3 weeks postoperatively are higher in subjects who develop clinical NEC (anticipating non-normal distribution of circulating iFABP based on preliminary data). ANOVA or non-parametric equivalents are used to assess whether severity by Bell staging is associated with iFABP levels, then Tukey's post-hoc test is used to interrogate whether Bell Stage 2/3 has circulating levels of iFABP significantly different from the lower levels. To assess whether iFABP decreases with therapy, a Wilcoxon signed-rank test was performed on pre/post therapy iFABP levels. Systems biology analysis was also pursued to perform a proteome-wide analysis. In a first approach, a Mann-Whitney U test was used for each protein measured in OLINK Explore 3072 measured at 7 and 14 days postoperatively to test for differences between NEC and no-NEC patients. Additionally, the effect of each protein measured in OLINK Explore 3072 is modeled across all time points on whether a patient develops NEC using a mixed effect regression model (i.e., the glmer function lme4 R-package). Mixed effect regression models are known to be able to readily handle both missing data and samples collected at irregular time intervals. P-values from these models are obtained using a likelihood ratio test via ANOVA and then FDR-corrected. Once a list of significant proteins is generated, the same network-based systems biology approach is used to determine significantly dysregulated biological processes and pathways as described above.

### Example 4—Specific Aim 3 Determining Dysregulated Proteins and Biologic Systems in the Early Postoperative and Convalescent Periods That Are Associated With Subsequent Neurodevelopmental Impairment As Measured By 1 Yr Bayley Scales of Infant and Toddler Development, 4Th Edition (BSID-4), Including Targeted Validation of an Embedded Panel of 8 A Priori Selected Neurologic Biomarkers (TNC, TIMD4, HO-1, OSMR, LTBP2, CTSL1, CRTAC1, and DCN)

In these experiments, Applicants validate the ability of 8 novel neurologic injury biomarkers (tenascin C [TNC], T-cell immunoglobulin and mucin domain-containing protein 4 [TIMD4], heme oxygenase-1 [HO-1], oncostatin M receptor beta [OSMR], latent transforming growth factor beta protein 2 [LTBP2], cartilage acidic protein 1 [CRTAC1], cathepsin L1 [CTSL1], and decorin [DCN]), alone and in combination, to differentiate between subjects with/without subsequent neurodevelopmental impairment. Additionally, Applicants measure the progressive postoperative changes in these biomarkers in order to define the natural history and identify the optimal biomarker screening period.

Applicants hypothesized that decreased CRTAC1 and elevated TNC, TIMD4, HO-1,OSMR, LTBP2, CTSL1, and DCN accurately predict decreased motor, language, and cognitive BSID-4 scores at lyr of age. The optimal timing and combinations of these biomarkers may vary, with biomarkers of central nervous system (CNS) injury/inflammation favoring screening in the early postoperative period and biomarkers of CNS repair favoring screening in the convalescent phase.

Applicants perform serial postoperative measurement of the circulating protcome to elucidate biologic pathways/protein networks associated with subsequent neurodevelopmental impairment, hypothesizing that higher levels of circulating markers of neurologic injury early after surgery and after any secondary neurologic insults are related to lower motor, language, and cognitive BSID-4 scores at 1 yr of age. Subjects with biomarker evidence of early CNS injury and subsequent normal recovery (normal 1 yr BSID-4) shows distinct proteomic patterns of repair compared to subjects with biomarker evidence of early CNS injury and subsequent neurodevelopmental delays.

Neurodevelopmental delay and disability are common after neonatal CHD surgery: Recent improvements in immediate postoperative outcomes have placed increased emphasis on long-term outcomes and quality of life. Neurodevelopmental delays and disabilities remain a particularly vexing problem. This issue is especially evident following high risk neonatal surgery where patients demonstrate a >50% overall prevalence of neurodevelopmental impairment, including >10% with severe impairment. This impairment begins early and has lifelong effects. Early outcomes of both the Boston Circulatory Arrest trial for transposition of the great arteries80 and the Single Ventricle Reconstruction Trial demonstrated neurodevelopmental outcomes significantly below population norms. These deficits continued on follow-up at grade-school age and into adulthood7 resulting in lower educational levels, unemployment, and reduced lifetime income. Thus, successful strategies to improve neurodevelopmental outcomes would have substantial impact on quality of life for patients with CHD.

Infancy represents a key window of risk and opportunity. While abnormal neurodevelopment begins prenatally in children with complex CHD, the plasticity and synaptic development of the infant period make the brain sensitive to modification, and the first year of life is a key window for determining neurodevelopmental outcomes. Unfortunately, for children with CHD this time period also involves repeated detrimental exposures. These include perioperative factors such as CPB, ECMO, low cardiac output, cyanosis, and thromboembolism as well as prolonged hospitalizations with a decrease in normal developmental activities and increased exposure to detrimental medications (e.g. general anesthetics, opioids, and benzodiazepines) and noxious stimuli. Mitigating injury and promoting neurodevelopment during this period could provide a lifetime of benefit for these high risk patients.

Disclosed methods and systems may be useful in diagnosing, risk stratifying, and treating various neurologic injuries in infancy, which have, until Applicant's present disclosure, remained limited. Neuro-developmental testing identifies delays in toddlers and school age children; however, it is less reliable in the neonatal period and is not a good stratification tool for early postoperative injury. Established clinical risk factors (e.g., prematurity, genetic disorders) explain only a portion of the variability and often are not modifiable. Easy to measure perioperative variables also account for only a small proportion (<5%) of the variability in neurodevelopmental outcomes. Socioeconomic factors may play an important role but still do not explain the majority of variation in neurodevelopmental outcomes. Neuroimaging by MRI consistently identifies both pre and postoperative changes in the brains of high risk neonates. Many of these changes, though, resolve with time and are not consistently related to longer-term outcomes. Also, MRI is generally a poor tool for serial monitoring in the neonatal period, especially for physiologically unstable patients. Circulating protein biomarkers could help fill this diagnostic gap by allowing serial bedside monitoring. To date, no biomarkers are clinically available to help risk stratify, diagnose or follow neurologic injury in CHD patients. Two protein biomarkers initially developed for adult CNS injury (glial fibrillary acidic protein and S100B) have shown modest association with neurodevelopmental outcomes in early observational studies. These markers have yet to be validated for clinical use.

Detection and analysis of circulating protein biomarkers serve two purposes directly related to improving patient outcomes. First, studies have shown that early interventions by psychologists and therapists may be beneficial, but these interventions are time intensive, costly, and inconsistently applied. Biomarkers capable of diagnosing and risk stratifying early neurologic injury could allow targeted application of these valuable human resources towards those children most likely to develop neurodevelopmental delays. Also, biomarker panels have the potential to help identify specific patient phenotypes most likely to respond to neurodevelopmental therapy. Second, therapies for acute neurologic injury are largely limited to prevention of catastrophic neurologic events and minimizing exposure to toxic medications/stimuli because little is known about the mechanisms driving neurologic injury and neurodevelopmental delay in CHD patients. Identification of protein systems activated during neurologic injury and recovery could provide key insights into the systemic mechanisms of injury and repair, paving the way for bedside to bench translational studies of novel mechanisms and therapeutic targets.

Here, Applicants describe performing both targeted validation of 8 novel biomarkers of neurologic injury as well as unbiased proteomic profiling of neurologic injury, recovery, and progression to neurodevelopmental delay in neonates undergoing open heart surgery.

Applicants, as part of a cohort study on the multi-omic response to staged surgical palliation, performed preliminary targeted proteomic profiling of 184 circulating proteins (OLINK Cardiometabolic and Cardiovascular II panels; Uppsala, Sweden; olink.com) in 18 infants with SVHD immediately prior to their Stage 2 surgery (Glenn or hemi-Fontan). The cohort demonstrated significantly decreased mean [SD] standardized scores in cognitive (88 [8.8]; p=0.0005), language (92 [18]; p=0.01), and motor (85 [12]; p<0.0001) skills compared to population norms. Infants in the lower quartile of motor scores (<78) showed a distinct proteomic fingerprint compared to infants in the upper quartile (>95) (FIG. 5). Differences were driven primarily by 9 proteins (p<0.05), 8 of which are involved in CNS injury/regeneration in animal models and adults: 1) TNC (extracellular matrix protein implicated in guidance of migrating neurons as well as axons during development), 2) CRTAC1 (axon formation and association with Alzheimer's Disease95), 3) TIMD4 (enhances engulfment of apoptotic CNS cells96), 4) HO-1 (mixed neuronal survival/degeneration, overexpression in Parkinson's disease97), 5) OSMR (combined neurotoxic and neuroprotective mechanisms through IL-6-like signaling, increases blood brain barrier permeability98), 6) CTSL1 (axonal growth99), 7) LTBP2 (assists in the folding and secretion of TGFB during neurologic injury and repair 100), and 8) DCN (small leucine proteoglycan expressed by neurons, astrocytes and Schwann cells, regulates axon growth 101). Area under the curve (AUC) for differentiating between upper and lower quartile motor scores ranged from 0.88 (CRTAC1) to 1.0 (TNC, TIMD4, HO-1). These preliminary data demonstrated the ability of proteomic profiling to identify novel, biologically relevant markers of neurologic injury in a highly efficient manner. Further study validates these biomarkers in a separate testing cohort and determines changes over time. Additionally, the proteomic panel used for the preliminary data was limited and enriched primarily with cardiovascular proteins. A more detailed analysis of the circulating proteome is useful in comprehensively evaluating markers of neurologic injury and allowing for evaluation of individual proteins and protein pathways/networks during the period of postoperative injury and recovery.

Here, Applicants describe validation of 8 candidate biomarkers of neurodevelopmental delay and discovery of additional novel biomarkers, including determination of optimal screening windows. Validated biomarkers assist identification of high-risk infants through a precision medicine approach, allowing early initiation of intensive physical, occupational, and speech therapy both in hospital and at home to optimize neurodevelopmental outcomes. These experiments provide for exploring candidate mechanisms of neurologic injury and recovery following neonatal CHD surgery using an unbiased/systems biology approach, identifying candidate targets for future translational, and interventional studies.

Much of the study design for the present experiments are the same as described above, as well as inclusion/exclusion criteria. Briefly, experiments take place in CHCO cardiac intensive care unit (ICU), stepdown unit, and neurodevelopmental care clinic, were BSID-4 cognitive, motor, and language standard scores at 1yr of age (+/−3 mo) are determined, as well as BSID-4 fine and gross motor and receptive and expressive language scaled scores at 1 year of age (+/−3 months); cardiac arrest; ECMO; stroke (hemorrhagic or thromboembolic); clinical seizures; confirmed genetic diagnosis. Applicants also investigate longitudinal plasma levels of CRTAC1, TNC, TIMD4, HO-1, OSMR, LTBP2, CTSL1, and DCN during the immediate postoperative period and in-hospital convalescence, along with daily plasma levels of CRTAC1, TNC, TIMD4, HO-1, OSMR, LTBP2, CTSL1, and DCN around acute clinical neurologic events; OLINK Explore 3072 proteomic profile (longitudinal and surrounding acute neurologic events).

Methods are substantially as described above, for example plasma samples (20 μL) are obtained preoperatively, and at 2, 24, and 48 hrs postoperatively (acute postoperative) and then weekly beginning on postoperative day 7 until discharge (convalescence). Samples are also collected daily for 5 days following acute episodes of potential neurologic injury (e.g., cardiac arrest, ECMO, stroke). As in Aim 2, convalescence samples use residual clinical samples to remove the need for repeated central line access or venipuncture after the acute postoperative period. Proteomic analysis of samples is performed using OLINK Explore 3072, measuring 3072 proteins (including CRTAC1, TNC, TIMD4, HO-1, OSMR, LTBP2, CTSL1, and DCN) as per above. BSID-4 testing is performed and followed-up in the case of any abnormal test results.

As disclosed above, Applicants have collected and stored longitudinal samples for 63 neonates, including daily samples for 9 acute neurologic events (cardiac arrest, ECMO, or stroke), and Applicants have operationalized lyr BSID-4 on a pilot basis for these subjects. Regarding preliminary analysis-of the 13 who have entered the testing age window, 9 have completed testing, 1 is pending, and 3 died during the follow-up period, demonstrating a high rate of successful follow-up (˜70%).

Power analysis involves power calculations performed based on preliminary data for the neurological injury biomarkers. The study is powered to detect a Pearson correlation coefficient different from zero. Power calculations were performed based on pairwise correlation analyses between each of the 8 biomarkers and the BSID-4 scores. A type I error rate of 0.05/8=0.0063 (Bonferroni correction) was used. Next two-sided tests were used and the observed Pearson correlation coefficients from the preliminary data. Given a sample size of 125, an excellent (>80%) power to detect associations for CRTAC1 (95%), TIMD4 (100%), HO-1 (100%), DCN (83%), and CTSL1 (99%) and a 76% power to detect an association between OSMR and BSID-4 score, and 60% power to detect an association for LTBP2.

Single linear regression is used to assess whether the eight a priori biomarkers of interest are associated with BSID-4 scores. Biomarker levels measured at two weeks postoperatively are used as the predictor and BSID-4 scores measured at one year of age as the response. Separate single linear regression models are fit for each protein, and significance assessed via t-tests of the slope parameter with a p-value cutoff of 0.05/8=0.0063 (Bonferroni correction). Multivariable linear regression is explored with the eight biomarkers of interest as predictors and BSID-4 scores as the response, adjusting for any clinical covariates that are associated with BSID-4 scores on univariate analysis including but not limited to sex, race/ethnicity, STAT surgical score, single ventricle physiology, known genetic diagnosis, CPB time, deep hypothermic circulatory arrest/selective cerebral perfusion time, cardiac arrest, and use of ECMO. Finally, linear mixed models are used to assess whether BSID-4 scores are associated with biomarkers measured across multiple time points. To perform proteome-wide analysis, the effect of each protein measured in OLINK Explore 3072 is modeled across all time points in predicting the BSID-4 score using a mixed effect regression model (i.e., the Imer function in the lme4 R-package). Mixed effect regression models are known to be able to readily handle both missing data and samples collected at irregular time intervals. P-values from these models are obtained using a likelihood ratio test via ANOVA and then FDR corrected. Once a list of significant proteins is generated, the same network-based systems biology approach, as described above, is used to determine significantly dysregulated biological processes and pathways.

All references disclosed herein, whether patent or non-patent, are hereby incorporated by reference as if each was included at its citation, in its entirety. In case of conflict between reference and specification, the present specification, including definitions, will control.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

The description of certain embodiments included herein is merely exemplary in nature and is in no way intended to limit the scope of the disclosure or its applications or uses. In the included detailed description of embodiments of the present systems and methods, reference is made to the accompanying drawings which form a part hereof, and which are shown by way of illustration specific to embodiments in which the described systems and methods may be practiced. These embodiments are described in sufficient detail to enable those skilled in the art to practice presently disclosed systems and methods, and it is to be understood that other embodiments may be utilized, and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The included detailed description is therefore not to be taken in a limiting sense, and the scope of the disclosure is defined only by the appended claims.

From the foregoing it will be appreciated that, although specific embodiments of the invention have been described herein for purposes of illustration, various modifications may be made without deviating from the spirit and scope of the invention.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly. the specification and drawings are to be regarded in an illustrative manner and are not intended to limit the scope of the appended claims.

1. A composition for diagnosing or predicting NEC comprising:
two or more molecules with affinity for a biomarker, wherein the biomarker is one or more of LRRN1, VASH1, NELL2, DNAJB8, IGFBP1, PTPRN2, AFP, CD74, BST2, CD99, KIR3DL1, RBP2, CES2, NCS1, C4BPB, RAB6A, CCL4, FABP2, FUCA1, REG3A, GGA1, ATXN10, MUC13, ODAM, ARID4B, CCL2, CLEC11A, ITGAM, INPP1, ICOSLG, LRP1, MMP12, GBP2, GZMB, EPO, CXCL9, LGALS4, VIM, GZMH, MFAP5, CD46, SSB, CTSS, JAM2, KIR2DL3, BGN, PDGFC, RAD23B, FAM3B, GALNT7, VAT1, HBEGF, MIA, TPT1, ILIR1, CD83, MLN, CD200, CLEC4D, CLEC4C, CXLC10, BTN2A1, TFF2, CTRC, EPHX2, CHEK2, MFAP3, DCN, LGALS1, THBD, CDH1, FCN2, IL6R, ILKAP, FOSB, MASP1, DRAXIN, ABHD14B, CD274, ADAM8, SCARF 2, RGMA, DSC2, AKT3, NTF4, TBLX1, LYPD8, LYAR, ITGB1BP1, GH2, EPHA2, LRRC25, ALPP, LAG3, KLK4, CD300LF, HBQ1, FURIN, FLT4, FGFR2, NECTIN4, P4HB, ACTN4, WAS, HLA-E, PARP1, NT5C3A, CASP2, OSM, HSD11B1, SLAMF7, IL32, CCL3, TRIM21, TRAF2, ALDH3A1, EDAR, KLRB1, DFFA, TGFB1, REG4, CXCL1, CEP43, IRAG2, TP53INP1, GLO1, GRK5, PDGFRA, SDC4, PRKAR1A, FADD, GDF2, FAM3C, CASP3, MB, PAM, SERPINE1, TGFB1, ITGB1, CD93, ATP6V1F, BAX, NDRG1, SIRT5, MDGA1, STAMBP, EREG, IMPA1, CRADD, TNR, NPM1, PRDX1, PSME1, LAYN, CDH15, VTA1, NSFL1C, MSR1, TFF1, CLEC14A, bin2, PARK7, CCT5, SFTPA2, CRACR2A, SUGT1, CHAC2, GNE, FMR1, CDC27, CEP20, TBC1D23, UBAC1, OMG, VPS37A, TMPRSS15, KIFBP, CD207, CNPY4, CALCOCO1, CPE, METAP2, PPY, CDC37, FGFBP1, FGF5, SMPDL3A, NPPB, AMY2B, GUSB, ICAM2, CELA3A, PROC, PAEP, and ASAH2.

2. The composition of claim 1, wherein the biomarker is one or more of LRRN1, VASHI, NELL2, DNAJB8, IGFBP1, PTPRN2, AFP, CD74, BST2, CD99, KIR3DL1, RBP2, CES2, NCS1, C4BPB, RAB6A, CCL4, FABP2, FUCA1, REG3A, GGA1, ATXN10, MUC13, ODAM, ARID4B, CCL2, CLEC11A, ITGAM, INPP1, ICOSLG, LRP1, and MMP12.

3. The composition of claim 1, wherein at least one biomarker is FABP2.

4. The composition of claim 3, wherein the molecule is selected from a nucleic acid, a peptide, or combinations thereof.

5. A device comprising the composition of claim 2, wherein the molecule is covalently attached to a surface of the device.

6. A device comprising the composition of claim 2, wherein the molecule comprises nucleic acids and amino acids.

7. A method for detecting one or more biomarkers, comprising:
contacting a sample with at least one molecule having affinity for the the biomarker, the biomarker selected from one or more of LRRN1, VASH, NELL2, DNAJB8, IGFBP1, PTPRN2, AFP, CD74, BST2, CD99, KIR3DL1, RBP2, CES2, NCS1, C4BPB, RAB6A, CCL4, FABP2, FUCA1, REG3A, GGA1, ATXN10, MUC13, ODAM, ARID4B, CCL2, CLEC11A, ITGAM, INPP1, ICOSLG, LRP1, MMP12, GBP2, GZMB, EPO, CXCL9, LGALS4, VIM, GZMH, MFAP5, CD46, SSB, CTSS, JAM2, KIR2DL3, BGN, PDGFC, RAD23B, FAM3B, GALNT7, VATI, HBEGF, MIA, TPT1, ILIR1, CD83, MLN, CD200, CLEC4D, CLEC4C, CXLC10, BTN2A1, TFF2, CTRC, EPHX2, CHEK2, MFAP3, DCN, LGALS1, THBD, CDH1, FCN2, IL6R, ILKAP, FOSB, MASP1, DRAXIN, ABHD14B, CD274, ADAM8, SCARF 2, RGMA, DSC2, AKT3, NTF4, TBLX1, LYPD8, LYAR, ITGB1BP1, GH2, EPHA2, LRRC25, ALPP, LAG3, KLK4, CD300LF, HBQ1, FURIN, FLT4, FGFR2, NECTIN4, P4HB, ACTN4, WAS, HLA-E, PARP1, NT5C3A, CASP2, OSM, HSD11B1, SLAMF7, IL32, CCL3, TRIM21, TRAF2, ALDH3A1, EDAR, KLRB1, DFFA, TGFB1, REG4, CXCL1, CEP43, IRAG2, TP53INP1, GLO1, GRK5, PDGFRA, SDC4, PRKAR1A, FADD, GDF2, FAM3C, CASP3, MB, PAM, SERPINE1, TGFB1, ITGB1, CD93, ATP6V1F, BAX, NDRG1, SIRT5, MDGA1, STAMBP, EREG, IMPA1, CRADD, TNR, NPM1, PRDX1, PSME1, LAYN, CDH15, VTA1, NSFL1C, MSR1, TFF1, CLEC14A, bin2, PARK7, CCT5, SFTPA2, CRACR2A, SUGT1, CHAC2, GNE, FMR1, CDC27, CEP20, TBC1D23, UBAC1, OMG, VPS37A, TMPRSS15, KIFBP, CD207, CNPY4, CALCOCO1, CPE, METAP2, PPY, CDC37, FGFBP1, FGF5, SMPDL3A, NPPB, AMY2B, GUSB, ICAM2, CELA3A, PROC, PAEP, and ASAH2.

8. The method of claim 7, wherein the biomarker is one or more of wherein the biomarker is selected from LRRN1, VASH1, NELL2, DNAJB8, IGFBP1, PTPRN2, AFP, CD74, BST2, CD99, KIR3DL1, RBP2, CES2, NCS1, C4BPB, RAB6A, CCL4, FABP2,FUCA1, REG3A, GGA1, ATXN10, MUC13, ODAM, ARID4B, CCL2, CLEC11A, ITGAM, INPP1, ICOSLG, LRP1, and MMP12.

9. The method of claim 8, wherein the amount of biomarker or molecule correlates to a signal, and the signal is higher or lower than in the sample compared to a control sample or compared to a sample taken at an earlier timepoint.

10. A system comprising the device of claim 2.

11. A method of for diagnosing or predicting NEC in a patient at risk thereof, comprising:
obtaining a sample of circulating proteins from the patient;
identifying the presence of one or more biomarkers in the sample to determine a biomarker signature; and
comparing the patient biomarker signature to a diagnostic and/or predictive signature to determine the presence of NEC and/or risk of developing NEC.

12. The method of claim 11, wherein the biomarker is one or more of LRRN1, VASH1, NELL2, DNAJB8, IGFBP1, PTPRN2, AFP, CD74, BST2, CD99, KIR3DL1, RBP2, CES2, NCS1, C4BPB, RAB6A, CCL4, FABP2, FUCA1, REG3A, GGA1, ATXN10, MUC13, ODAM, ARID4B, CCL2, CLEC11A, ITGAM, INPP1, ICOSLG, LRP1, MMP12, GBP2, GZMB, EPO, CXCL9, LGALS4, VIM, GZMH, MFAP5, CD46, SSB, CTSS, JAM2, KIR2DL3, BGN, PDGFC, RAD23B, FAM3B, GALNT7, VAT1, HBEGF, MIA, TPT1, ILIR1, CD83, MLN, CD200, CLEC4D, CLEC4C, CXLC10, BTN2A1, TFF2, CTRC, EPHX2, CHEK2, MFAP3, DCN, LGALS1, THBD, CDH1, FCN2, IL6R, ILKAP, FOSB, MASP1, DRAXIN, ABHD14B, CD274, ADAM8, SCARF 2, RGMA, DSC2, AKT3, NTF4, TBLX1, LYPD8, LYAR, ITGB1BP1, GH2, EPHA2, LRRC25, ALPP, LAG3, KLK4, CD300LF, HBQ1, FURIN, FLT4, FGFR2, NECTIN4, P4HB, ACTN4, WAS, HLA-E, PARP1, NT5C3A, CASP2, OSM, HSD11B1, SLAMF7, IL32, CCL3, TRIM21, TRAF2, ALDH3A1, EDAR, KLRB1, DFFA, TGFB1, REG4, CXCL1, CEP43, IRAG2, TP53INP1, GLO1, GRK5, PDGFRA, SDC4, PRKAR1A, FADD, GDF2, FAM3C, CASP3, MB, PAM, SERPINE1, TGFB1, ITGB1, CD93, ATP6V1F, BAX, NDRG1, SIRT5, MDGA1, STAMBP, EREG, IMPA1, CRADD, TNR, NPM1, PRDX1, PSME1, LAYN, CDH15, VTA1, NSFL1C, MSR1, TFF1, CLEC14A, bin2, PARK7, CCT5, SFTPA2, CRACR2A, SUGT1, CHAC2, GNE, FMRI, CDC27, CEP20, TBC1D23, UBAC1, OMG, VPS37A, TMPRSS15, KIFBP, CD207, CNPY4, CALCOCO1, CPE, METAP2, PPY, CDC37, FGFBP1, FGF5, SMPDL3A, NPPB, AMY2B, GUSB, ICAM2, CELA3A, PROC, PAEP, and ASAH2.

13. The method of claim 12, wherein the biomarker is one or more of wherein the biomarker is selected from LRRN1, VASH1, NELL2, DNAJB8, IGFBP1, PTPRN2, AFP, CD74, BST2, CD99, KIR3DL1, RBP2, CES2, NCS1, C4BPB, RAB6A, CCL4, FABP2, FUCA1, REG3A, GGA1, ATXN10, MUC13, ODAM, ARID4B, CCL2, CLEC11A, ITGAM, INPP1, ICOSLG, LRP1, and MMP12.

14. The method of claim 13, wherein the biomarker is FABP2.

15. The method of claim 15, where in the signal demonstrates that at least one biomarker's expression is downregulated.

16. The method of claim 15, wherein the biomarker one or more of LRRN1, VASH1, NELL2, DNAJB8, IGFBP1, PTPRN2, AFP, CD74, BST2, CD99, KIR3DL1, RBP2, CES2, NCS1, RAB6A, CCL4, FABP2, REG3A, GGA1, ATXN10, MUC13, ODAM, ARID4B, CCL2, CLEC11A, ITGAM, INPP1, ICOSLG, LRP1, MMP12, GBP2, GZMB, EPO, CXCL9, LGALS4, VIM, GZMH, CD46, SSB, CTSS, JAM2, KIR2DL3, BGN, RAD23B, GALNT7, VAT1, HBEGF, MIA, TPT1, ILIR1, CD83, MLN, CD200, CLEC4D, CLEC4C, CXLC10, BTN2A1, TFF2, EPHX2, CHEK2, MFAP3, DCN, LGALS1, THBD, CDH1, FCN2, ILKAP, FOSB, MASP1, DRAXIN, ABHD14B, CD274, ADAM8, SCARF 2, RGMA, AKT3, TBLX1, LYPD8, LYAR, ITGB1BP1, GH2, EPHA2, LRRC25, ALPP, LAG3, KLK4, CD300LF, HBQ1, FURIN, FLT4, FGFR2, NECTIN4, P4HB, ACTN4, WAS, HLA-E, PARP1, NT5C3A, CASP2, OSM, HSD11B1, SLAMF7, IL32, CCL3, TRIM21, TRAF2, ALDH3A1, EDAR, KLRB1, DFFA, TGFB1, REG4, CXCL1, CEP43, IRAG2, TP53INP1, GLO1, GRK5, PDGFRA, SDC4, PRKAR1A, FADD, GDF2, FAM3C, CASP3, MB, PAM, SERPINE1, TGFBI, ITGB1, CD93, ATP6V1F, BAX, NDRG1, SIRT5, MDGA1, STAMBP, EREG, IMPA1, CRADD, TNR, NPM1, PRDX1, PSME1, LAYN, CDH15, VTA1, NSFL1C, MSR1, TFF1, CLEC14A, bin2, PARK7, CCT5, SFTPA2, CRACR2A, SUGT1, CHAC2, GNE, FMR1, CDC27, CEP20, TBC1D23, UBAC1, OMG, VPS37A, TMPRSS15, KIFBP, CD207, CNPY4, CALCOCO1, CPE, METAP2, PPY, CDC37, FGFBP1, FGF5, NPPB, AMY2B, ICAM2, PROC, PAEP, and ASAH2.

17. The method of claim 12, where in the signal demonstrates that at least one biomarker's expression is upregulated.

18. The method of claim 17, wherein the biomarker is one or more of C4BPB, FUCA1, MFAP5, PDGFC, HMOX1, LBP, ILIRL2, TPP1, FAM3B, CTRC, IL6R, DSC2, NTF4, SMPDL3A, GUSB, and CELA3A.

19. The method of claim 13, where in the signal demonstrates that at least one biomarker's expression is upregulated and the biomarker is one or more of C4BPB, FUCA1, MFAP5, PDGFC, HMOX1, LBP, ILIRL2, TPP1, FAM3B, CTRC, IL6R, DSC2, NTF4, SMPDL3A, GUSB, and CELA3A.

20. The method of claim 14, where in the signal demonstrates that at least one biomarker's expression is upregulated and the biomarker is one or more of C4BPB, FUCA1, MFAP5, PDGFC, HMOX1, LBP, ILIRL2, TPP1, FAM3B, CTRC, IL6R, DSC2, NTF4, SMPDL3A, GUSB, and CELA3A
